(19) United States
(12) Patent Application Publication
(10) Pub. No.: US 2026/0240965 A1
(43) Pub. Date: Aug. 20, 2026

(54) IMMUNOGENIC PEPTIDES OF FUNGAL PATHOGENS AND USES THEREOF

(71) Applicant: University of Georgia Research Foundation, Inc., Athens, GA (US)

(72) Inventors: Karen Norris, Athens, GA (US); Emily Rayens, Athens, GA (US); Whitney Rabacal, Athens, GA (US)

(73) Assignee: University of Georgia Research Foundation, Inc., Athens, GA (US)

(21) Appl. No.: 19/655,481

(22) Filed: Apr. 22, 2026

Related U.S. Application Data
(63) Continuation of application No. PCT/US2024/053890, filed on Oct. 31, 2024.
(60) Provisional application No. 63/547,187, filed on Nov. 3, 2023.

(51) Int. Cl. A61K 39/00; A61P 37/04; C07K 14/37; C07K 14/39; C07K 14/40

(52) U.S. Cl. CPC A61K 39/0002; A61P 37/04; C07K 14/37; C07K 14/39; C07K 14/40

(57) ABSTRACT

Described are recombinant immunogenic kex peptide antigens, recombinant peptide immunogens, immunogenic compositions, and methods of using such immunogens and compositions for the treatment of and protection against diseases and symptoms thereof associated with infection by or exposure to one or more fungal pathogens. Also provided are compositions and kits for detecting or quantifying the presence of antibodies directed against one or more fungal kex peptide antigens, as well as use of such antibodies to treat and protect against diseases and symptoms thereof associated with infection by or exposure to one or more fungal pathogens.

### CROSS REFERENCE TO RELATED APPLICATIONS

This application is a continuation under 35 U.S.C. § 111 (a) of PCT International Patent Application No. PCT/US2024/053890, filed Oct. 31, 2024, designating the United States and published in English, which claims priority to and the benefit of U.S. Provisional Application No. 63/547,187, filed Nov. 3, 2023, the entire contents of each of which are incorporated by reference herein.

### SEQUENCE LISTING

The instant application contains a Sequence Listing which has been submitted electronically in XML file format and is hereby incorporated by reference in its entirety. The XML copy, created on Oct. 28, 2024, is named 173093-011801_PCT SL.xml and is 14,761 bytes in size.

### BACKGROUND

Pathogenic fungal organisms are universal in the environment and can cause global health threats. Pathogenic fungi are typically not harmful to individuals with healthy and normally functional immune systems even after exposure, for example, by inhalation. However, individuals with weakened or compromised immune systems, or those having pre-existing medical conditions, such as those with lung diseases or viral infections, such as HIV/AIDS infection, are at a higher risk of developing serious health problems and adverse reactions following exposure to and infection by fungal pathogens.

Because of the grave repercussions of infection by fungal organisms in individuals in poor medical health and in those with weakened immune systems, there is an ongoing and urgent need for methods and compositions for treating or preventing infection and associated diseases caused by or associated with these pathogens. Cost-effective and efficient methods of treatment of and protection from fungal pathogens are needed, particularly in less affluent parts of the world. Effective methods and approaches for treating or preventing diseases caused by or associated with infection by fungal pathogens for which suitable therapies are currently nonexistent or inadequate are urgently needed to alleviate fungal pathogens that pose ever-present threats to at-risk individuals worldwide.

### SUMMARY

Immunogenic kexin polypeptides and, in particular, kex (kex1) peptide antigens, compositions containing such polypeptides and peptide antigens, and methods of use are described and featured herein. Methods for treating, protecting against, or preventing diseases, pathologies, and/or the symptoms thereof, caused by or associated with infection (e.g., opportunistic infection) by fungal pathogens are provided and described herein. In an embodiment, the immunogenic kex peptide antigen is an isolated, recombinant peptide or is recombinantly produced, e.g. a non-naturally occurring, recombinant, immunogenic kex peptide antigen.

In an aspect, an immunogenic peptide antigen, or a polynucleotide encoding the antigen, having at least or at least about 85%, 90%, 95%, 96%, 97%, 98%, 99%, or greater amino acid sequence identity to one or more of the following amino acid sequences is provided:

DDGMTMEGPGTLIQRAFVNGIQQGRAGRGSIYVFAAGNGALHEDNCNFD
GYTNSIYSVTVGAVDRDDNHPYYSESCSAMLVVTYSSG
(Histoplasma capsulatum HC.KEX1 (EER45430.1,
residue numbers 316 to 402), (SEQ ID NO: 1));
DNGATMDAPGLLIRRAMVHGIQQGRGGKGSIFVFAAGNGAASGDNCNFD
GYTNSIYSITVGAIDREDKHPYYSESCSAQLVVTYSSG
(Coccidioides immitis CI.KEX1 (XP_001246854.1),
(SEQ ID NO: 2));
PDNGQTMEAPNGILADAFINGIKKGRGGKGSIYVFATGNGATSGDNCNF
DGYTNSIYTITVGAIDHRNEHPPYSESCSAQLVVTYSSGGG
(Mucor circinelloides MC.KEX1 (from EPB86942.1,
residue numbers 290 to 378), (SEQ ID NO: 3));
PDNGENMEAPKAILTDAIANGVRNGRDGKGSIYVFATGNGATLGDNCNF
DAYTNSIYTITVGAIDHTNKHPAYSESCSAQLVVTYSSGSG
(Rhizopus oryzae RO.KEX1 (KAG1151579.1, residue
numbers 289-378), (SEQ ID NO: 4)), (also called
Rhizopus arrhizus);
PDLGEVAEGPQGIILDAIRNGIENGRQGKGSIFVFASGNGGHNDDNCNF
DGYTNSIYTITVGAIDRLGNYPSYAEKCAAQFFVTYSSGSG
(Rhizopus delemar RD.KEX1 (KAG1464495.1, residue
numbers 742-831), (SEQ ID NO: 5));
and
DNGKTVAAPDMLVRKAMIKGIQDGRQGKGAVYVFASGNGRQYDDQCNFD
GYTNSIYSITVGAIDHQNQWPFYAEACSAVMVVTYSAGSG
(Candida auris CAu.KEX1 (KND99254.2, residue
numbers 304 to 393), (SEQ ID NO: 6))

The above-described sequences reflect kex (kex1) peptide amino acid sequences derived from the fungal organisms as indicated, such as Histoplasma capsulatum, Coccidioides immitis, Mucor circinelloides, Rhizopus oryzae (also called Rhizopus arrhizus), Rhizopus delemar, and Candida auris.

In an aspect, an immunogenic peptide antigen, or a polynucleotide encoding the antigen, comprising or consisting of one or more of the above-described amino acid sequences is provided. The terms “immunogenic polypeptide,” “immunogenic peptide,” “immunogenic polypeptide antigen” and “immunogenic peptide antigen” are used interchangeably herein.

In an aspect, an isolated, recombinant immunogenic peptide antigen or a polynucleotide encoding the peptide antigen having at least about 95% or more amino acid sequence identity to one or more of

DDGMTMEGPGTLIQRAFVNGIQQGRAGRGSIYVFAAGNGALHEDNCNFD
GYTNSIYSVTVGAVDRDDNHPYYSESCSAMLVVTYSSG as set
forth in SEQ ID NO: 1;
DNGATMDAPGLLIRRAMVHGIQQGRGGKGSIFVFAAGNGAASGDNCNFD
GYTNSIYSITVGAIDREDKHPYYSESCSAQLVVTYSSG as set
forth in SEQ ID NO: 2;
PDNGQTMEAPNGILADAFINGIKKGRGGKGSIYVFATGNGATSGDNCNF
DGYTNSIYTITVGAIDHRNEHPPYSESCSAQLVVTYSSGGG as set
forth in SEQ ID NO: 3;
PDNGENMEAPKAILTDAIANGVRNGRDGKGSIYVFATGNGATLGDNCNF
DAYTNSIYTITVGAIDHTNKHPAYSESCSAQLVVTYSSGSG as set
forth in SEQ ID NO: 4;
PDLGEVAEGPQGIILDAIRNGIENGRQGKGSIFVFASGNGGHNDDNCNF
DGYTNSIYTITVGAIDRLGNYPSYAEKCAAQFFVTYSSGSG as set
forth in SEQ ID NO: 5;
and
DNGKTVAAPDMLVRKAMIKGIQDGRQGKGAVYVFASGNGRQYDDQCNFD
GYTNSIYSITVGAIDHQNQWPFYAEACSAVMVVTYSAGSG as set
forth in SEQ ID NO: 6, or immunogenic portions or
fragments thereof, is provided.

In an aspect, an isolated, recombinant immunogenic peptide antigen or a polynucleotide encoding the peptide antigen comprising or consisting of one or more of

DDGMTMEGPGTLIQRAFVNGIQQGRAGRGSIYVFAAGNGALHEDNCNFD
GYTNSIYSVTVGAVDRDDNHPYYSESCSAMLVVTYSSG as set
forth in SEQ ID NO: 1;
DNGATMDAPGLLIRRAMVHGIQQGRGGKGSIFVFAAGNGAASGDNCNFD
GYTNSIYSITVGAIDREDKHPYYSESCSAQLVVTYSSG as set
forth in SEQ ID NO: 2;
PDNGQTMEAPNGILADAFINGIKKGRGGKGSIYVFATGNGATSGDNCNF
DGYTNSIYTITVGAIDHRNEHPPYSESCSAQLVVTYSSGGG as set
forth in SEQ ID NO: 3;
PDNGENMEAPKAILTDAIANGVRNGRDGKGSIYVFATGNGATLGDNCNF
DAYTNSIYTITVGAIDHTNKHPAYSESCSAQLVVTYSSGSG as set
forth in SEQ ID NO: 4;
PDLGEVAEGPQGIILDAIRNGIENGRQGKGSIFVFASGNGGHNDDNCNF
DGYTNSIYTITVGAIDRLGNYPSYAEKCAAQFFVTYSSGSG as set
forth in SEQ ID NO: 5;
and
DNGKTVAAPDMLVRKAMIKGIQDGRQGKGAVYVFASGNGRQYDDQCNF
DGYTNSIYSITVGAIDHQNQWPFYAEACSAVMVVTYSAGSG as set
forth in SEQ ID NO: 6, or immunogenic portions or
fragments thereof, is provided.

In another aspect, an immunogenic composition comprising an effective amount of the immunogenic peptide antigen of either of the above-delineated aspects, or a polynucleotide encoding the peptide antigen, and a pharmaceutically acceptable carrier or excipient is provided. In an embodiment, the immunogenic composition further comprises an adjuvant. In an embodiment, the adjuvant is alpha-galactosylceramide (αGC). In an embodiment, the adjuvant is alum.

In another aspect, an immunogenic vaccine comprising the isolated, recombinant immunogenic peptide antigen of either of the above-delineated aspects, or a polynucleotide encoding the peptide antigen is provided.

In embodiments of the above, a portion of the one or more immunogenic peptide antigens that has functional activity is provided. In an embodiment, the immunogenic peptide antigens as described above are recombinantly produced or are recombinant peptide antigens. In an embodiment, the recombinantly produced or recombinant peptide antigen is isolated, or is isolated and purified.

In another aspect, a method of eliciting an immune response in a subject is provided, in which the method involves administering to the subject the isolated, recombinant immunogenic peptide antigen of the above-delineated aspects and/or embodiments, or a polynucleotide encoding the peptide antigen, or a pharmaceutically acceptable composition thereof.

In another aspect, a method of eliciting an immune response in a subject is provided, in which the method involves administering to the subject the immunogenic composition of any of the above-delineated aspects and/or embodiments thereof.

In another aspect, a method of eliciting an immune response in a subject is provided, in which the method involves administering to the subject the immunogenic vaccine of the above-delineated aspects and/or embodiments thereof.

In embodiments of the above-delineated methods and/or embodiments thereof, the immune response elicited in the subject comprises the generation or production of antibodies directed against one or more of the isolated, recombinant immunogenic peptide antigens of the above-delineated aspects and/or embodiments.

In another aspect, a method of treating or protecting a subject against a disease and/or symptoms thereof, associated with or caused by infection by a fungal pathogen is provided, in which the method involves administering to the subject an effective amount of the isolated, recombinant immunogenic peptide antigen of the above-delineated aspects and/or embodiments, a polynucleotide encoding the peptide antigen, or a pharmaceutically acceptable composition thereof.

In another aspect, a method of treating or protecting a subject against a disease and/or symptoms thereof, associated with or caused by infection by a fungal pathogen is provided, in which the method involves administering to the subject an effective amount of the immunogenic composition of any of the above-delineated aspects and/or embodiments thereof.

In another aspect, a method of treating or protecting a subject against a disease and/or symptoms thereof, associated with or caused by infection by a fungal pathogen is provided, in which the method involves administering to the subject an effective amount of the immunogenic vaccine of the above-delineated aspect and/or an embodiment thereof.

In another aspect, a method of treating or protecting a subject against disease and/or symptoms thereof associated with infection by a fungal pathogen is provided, in which the method involves administering to the subject an immunogenic peptide of SEQ ID NO: 7, a polynucleotide encoding the peptide, or a pharmaceutical composition comprising the peptide or the polynucleotide, wherein the peptide elicits in the subject an immune response comprising antibodies that cross-react with a kex peptide antigen of one or more of SEQ ID NOs: 1-6 produced by different fungal pathogens. In an embodiment of the method, the fungal pathogen is selected from one or more of Histoplasma capsulatum, Coccidioides immitis, Mucor circinelloides, Rhizopus oryzae, Rhizopus delemar, or Candida auris.

In another aspect, a method of treating or protecting a subject against disease and/or symptoms thereof associated with infection by a fungal pathogen is provided, in which the method involves administering to the subject a recombinant, immunogenic peptide of any one of SEQ ID NOs: 1-6, a polynucleotide encoding the peptide, or a pharmaceutical composition comprising the peptide or the polynucleotide, wherein the recombinant immunogenic peptide elicits in the subject an immune response comprising antibodies that bind to the kex peptide antigen of one or more of SEQ ID NOs: 1-6, produced by the Histoplasma capsulatum, Coccidioides immitis, Mucor circinelloides, Rhizopus oryzae, Rhizopus delemar, or Candida auris fungal pathogens.

In another aspect, an isolated antiserum comprising an antibody that specifically binds to one or more of the immunogenic peptide antigens of the above-delineated aspects and/or embodiments thereof, or an antigen-binding fragment thereof, is provided, in an amount effective to treat or protect a subject against disease and/or a symptom thereof associated with infection by one or more of Histoplasma capsulatum, Coccidioides immitis, Mucor circinelloides, Rhizopus oryzae, Rhizopus delemar, or Candida auris fungal pathogens.

In an aspect, an isolated antiserum comprising an antibody that specifically binds to one or more of the immunogenic peptide antigens of the above-delineated aspects and/or embodiments thereof, or an antigen-binding fragment thereof, is provided.

In an embodiment of the above-described aspects directed to the isolated antiserum, the antiserum is generated by immunizing a subject with pan-fungal peptide 2 of SEQ ID NO: 7.

In an aspect, an isolated or purified antibody or an antigen-binding fragment that specifically binds to an immunogenic peptide of the above-delineated aspects and/or embodiments thereof is provided, in an amount effective to treat or protect a subject against disease and/or a symptom thereof associated with fungal infection by one or more of Histoplasma capsulatum, Coccidioides immitis, Mucor circinelloides, Rhizopus oryzae, Rhizopus delemar, or Candida auris.

In an aspect of any of the above-delineated methods or of any of the isolated antiserum or antibody and/or embodiments thereof, the antibody is a monoclonal antibody, a polyclonal antibody, or an antigen-binding fragment thereof.

In embodiments of the above-delineated methods and/or embodiments thereof, or in embodiments of the above-delineated isolated antiserum or antibody and/or embodiments thereof, the method, isolated antiserum or antibody treats or protects against one or more diseases or pathologies selected from thrush, invasive candidiasis, lung or pulmonary disease or dysfunction, pneumonia-like illness, meningitis, histoplasmosis, flu-like disease, fever, chills, headache, cough, fatigue, shortness of breath, muscle aches, rash, coddidioidomycosis or valley fever, cutaneous infection, zygomycosis, and symptoms thereof.

In another aspect, a method of treating or protecting a subject against disease and/or a symptom thereof associated with fungal infection is provided, in which the method involves administering to the subject an effective amount of an antibody or an antigen-binding fragment thereof that specifically binds to one or more of SEQ ID NOs: 1-6.

In an embodiment of the above-delineated methods and/or embodiments thereof, the antibody or fragment thereof is present in or isolated from an antiserum derived from a donor subject. In an embodiment of the above-delineated methods and/or embodiments thereof, the antibody or an antigen-binding fragment thereof specifically binds an immunogenic kex peptide antigen of one or more fungal pathogens selected from Histoplasma capsulatum, Coccidioides immitis, Mucor circinelloides, Rhizopus oryzae, Rhizopus delemar, or Candida auris.

In an aspect, antisera generated or produced by immunizing a subject with the recombinant, immunogenic peptide comprising or consisting of the amino acid sequence PDDGKTMEGPDILVLRAFINGVQNGRDGKGSIYVFASGNGGGFEDNCNFDGYTNSIY SITVGAIDRKGLHPSYSEACSAQLVVTYSSGSG (Pan-fungal peptide 2), (SEQ ID NO: 7) contain antibodies that bind to and/or react (cross-react) with one or more of the above-described immunogenic recombinant peptide antigens of SEQ ID NOs: 1-6.

In another aspect, method of treating or protecting a subject against disease or disease severity associated with fungal infection is provided, in which the method involves administering to the subject an immunogenic composition comprising an immunogenic recombinant kex peptide antigen of one or more of SEQ ID NOs: 1-6, an immunogenic portion or fragment thereof, or a polynucleotide encoding one of more of SEQ ID NOs: 1-6, or an immunogenic portion or fragment thereof, in an amount effective to induce an immune response against a fungal pathogen, or more than one fungal pathogen, such as, for example, Candida, Cryptococcus, Histoplasma, Coccidioides, Mucor, Rhizopus, Pneumocystis, and/or Aspergillus fungal pathogens.

In an embodiment of the above-delineated methods and/or embodiments thereof, or in an embodiment of the above-delineated isolated antiserum or antibody and/or embodiments thereof, the subject is a non-human mammal or human subject. In an embodiment of the foregoing, the subject is immunocompromised or immunosuppressed. In an embodiment, the immunocompromised or immunosuppressed subject is a pre-transplant subject or a post-transplant subject. In an embodiment, the immunocompromised or immunosuppressed subject is being treated for cancer, an immunodeficiency disease, a congenital disease, or an autoimmune disease. In an embodiment, the immunocompromised or immunosuppressed subject is being treated for HIV, rheumatoid arthritis, or psoriasis. In an embodiment of the above-delineated methods and/or embodiments thereof, an adjuvant is administered to the subject. In an embodiment, the adjuvant is alpha-galactosylceramide (α-GC) or alum.

In another aspect, a method of killing or enhancing killing of Candida auris fungal cells is provided in which the method involves contacting the Candida auris fungal cells with antibodies generated against an immunogenic recombinant peptide antigen comprising or consisting of DNGKTVAAPDMLVRKAMIKGIQDGRQGKGAVYVFASGNGRQYDDQCNFDGYTNSIYSITVGA IDHQNQWPFYAEACSAVMVVTYSAGSG as set forth in SEQ ID NO: 6, or with an antiserum containing said antibodies, in an amount effective to kill the cells.

In another aspect, a method of killing or enhancing killing of Histoplasma capsulatum fungal cells is provided in which the method involves contacting the Histoplasma capsulatum fungal cells with antibodies generated against an immunogenic recombinant peptide antigen comprising or consisting of DDGMTMEGPGTLIQRAFVNGIQQGRAGRGSIYVFAAGNGALHEDNCNFDGYTNSIYSVTVGA VDRDDNHPYYSESCSAMLVVTYSSG as set forth in SEQ ID NO: 1, or with an antiserum containing said antibodies, in an amount effective to kill the cells.

In another aspect, a method of killing or enhancing killing of Coccidioides immitis fungal cells is provided in which the method involves contacting the Coccidioides immitis fungal cells with antibodies generated against an immunogenic recombinant peptide antigen comprising or consisting of DNGATMDAPGLLIRRAMVHGIQQGRGGKGSIFVFAAGNGAASGDNCNFDGYTNSIYSITVGA IDREDKHPYYSESCSAQLVVTYSSG as set forth in SEQ ID NO: 2, or with an antiserum containing said antibodies, in an amount effective to kill the cells.

In another aspect, a method of killing or enhancing killing of Mucor circinelloides fungal cells is provided in which the method involves contacting the Mucor circinelloides fungal cells with antibodies generated against an immunogenic recombinant peptide antigen comprising or consisting of PDNGQTMEAPNGILADAFINGIKKGRGGKGSIYVFATGNGATSGDNCNFDGYTNSIYTITVG AIDHRNEHPPYSESCSAQLVVTYSSGGG as set forth in SEQ ID NO: 3, or with an antiserum containing said antibodies, in an amount effective to kill the cells.

In another aspect, a method of killing or enhancing killing of Rhizopus oryzae fungal cells is provided in which the method involves contacting the Rhizopus oryzae fungal cells with antibodies generated against an immunogenic recombinant peptide antigen comprising or consisting of PDNGENMEAPKAILTDAIANGVRNGRDGKGSIYVFATGNGATLGDNCNFDAYTNSIYTITVG AIDHTNKHPAYSESCSAQLVVTYSSGSG as set forth in SEQ ID NO: 4, or with an antiserum containing said antibodies, in an amount effective to kill the cells.

In another aspect, a method of killing or enhancing killing of Rhizopus delemar fungal cells is provided in which the method involves contacting the Rhizopus delemar fungal cells with antibodies generated against an immunogenic recombinant peptide antigen comprising or consisting of PDLGEVAEGPQGIILDAIRNGIENGRQGKGSIFVFASGNGGHNDDNCNFDGYTNSIYTITVG AIDRLGNYPSYAEKCAAQFFVTYSSGSG as set forth in SEQ ID NO: 5, or with an antiserum containing said antibodies, in an amount effective to kill the cells.

In an embodiment of the above-delineated fungal cell killing methods, the antibodies or the antiserum containing the antibodies are generated in a subject infected with or exposed to the respective fungal pathogen. In an embodiment of the fungal cell killing methods, the contacting is in vitro, ex vivo, or in vivo. In an embodiment of these methods, the killing of the fungal cells is effected by phagocytic macrophages, e.g., as described in Example 9 herein.

Other features and advantages of the described embodiments will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in the embodiments described herein: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “kex,” kex1,” or “kexin” protein is meant a polypeptide or functional portion or fragment thereof, or a kex (kex1) peptide, having at least about 85% or greater amino acid identity to the amino acid sequence provided at GenBank Accession No. EU918304.1, at NCBI Accession No. XM_746441.1, at GenBank Accession No. AF022372.1, or at NCBI Accession No. XM_572303.1 and having immunogenic activity. In an embodiment, the kex peptide is an antigenically stable active site peptide sequence. (Kutty, G. and Kovacs, J. A., 2003, Infect. Immun., 71(1):571-574; Lee. L. H. et al., 2000, Gene, 242(1-2):141-150; and Russian, D. A. et al., 1999, Proc. Assoc. Am. Physicians, 111(4):347-356). In one embodiment, a kex or kex1 peptide is a fragment or portion of a naturally occurring kexin protein or is a non-naturally occurring pan-fungal peptide or fragment thereof. In an embodiment, the kex (kex1) peptide constitutes an antigen that is immunogenic (an immunogenic peptide antigen) when used as an immunogen that is administered to an animal. In an embodiment, the kex (kex1) peptide antigen is recombinant or recombinantly produced.

By “Pan-fungal peptide 2” is meant a kex peptide comprising a sequence having 95, 96, 97, 98, or 99% identity to Pan-fungal peptide 2 and having immunogenic activity. In some embodiments, a Pan-fungal peptide 2 comprises 1, 2, or 3 additional amino acids at the carboxy and/or amino terminus, which do not change the peptide's immunogenicity. The sequence of Pan-fungal peptide 2 follows:

(SEQ ID NO: 7)
PDDGKTMEGPDILVLRAFINGVQNGRDGKGSIYVFASGNGGGFEDNCNF
DGYTNSIYSITVGAIDRKGLHPSYSEACSAQLVVTYSSGSG
(Pan-fungal peptide 2),

An exemplary kex1 polypeptide fragment of Pneumocystis isolated from Pneumocystis colonized non-human primates (cynomolgus macaques) and having GenBank Accession No. EU918304.1 is provided below:

| (SEQ ID NO: 8) | |
|---|---|
| 1 | DDDGKTVDGP SPLVLRAFIN GVNNGRNGLG SIYVFASGNG GIYDDNCNFD GYANSVFTIT |
| 61 | IGGIDKHGKR FAYSEACSSQ LAVTYAGGSA |

An exemplary polynucleotide sequence encoding the kex1 polypeptide fragment provided at GenBank Accession No. EU918304.1 is provided below:

| (SEQ ID NO: 9) | |
|---|---|
| 1 | gatgacgatg gaaaaaccgt tgatgggcct tctcctcttg ttcttagagc atttattaat |
| 61 | ggagtaaata atgggaggaa tgggttgggt tctatctatg tttttgcatc aggaaatggc |
| 121 | ggaatatacg atgacaactg taattttgat ggatatgcaa atagcgtgtt tactattact |
| 181 | attggtggta tagataaaca cggaaagcgc tttgcatatt ctgaagcgtg ttcttctcag |
| 241 | ttagctgtta catatgcagg cggaagtgca |

An exemplary kex (kexB endoprotease) polypeptide sequence of Aspergillus fumigatus (Af293) having NCBI Accession No. XM_746441.1 is provided below:

(SEQ ID NO: 10)
MRFLGSIALVLSSISVASANVRSRSYDTHEFFALHLDDSASPSHVAQLL
GARHEGQIGELANHHTFSIPRERSSDLDALLERARAARKIRRRARDDAT
SQEQHNDALGGILWSQKLAPKKRLVKRVPPPERLARTFATGKEDPVAAQ
SQKRIASTLGITDPIFNGQWHLFNTVQLGHDLNVTGVWMEGITGKGVTT
AVVDDGLDMYSNDLKPNYFPEGSYDFNDHTPEPRPRLSDDKHGTRCAGE
IAAARNDVCGVGVAYDSRVAGVRILSKAIDDADEATAINFAYQENDIFS
CSWGPPDDGATMEGPGILIKRAFVNGVQNGRGGKGSIFVFAAGNGASFE
DNCNFDGYTNSIYSITVGAIDREGNHPSYSESCSAQLVVAYSSGSGDAI
HTTDVGTDKCYSFHGGTSAAGPLAAGTVALALSARPELTWRDAQYLMVE
TAVPIHEDDGSWQVTKAGRKFSHDWGYGKVDAYALVQKAKTWELVKPQA
WFHSPWLRVQHKVPQGDQGLASSYEVTEQMMKNANIARLEHVTVTMNVN
HTRRGDLSVELRSPEGIVSHLSTTRKSDNEKAGYVDWTFMTVAHWGESG
VGRWTVIVKDTNVNEFTGEFIDWRLNLWGEAIDGANQKPHPFPDEHDDD
HSIEDAIVATTSVETGPTKTGVPGSTDDTINRPVNAKPVETQTPSPAET
TATKLAPPAETRPAATATSSPTPPAASDSFLPSFMPTFGASKRTQIWIY
AAIGSIIVFCIGLGIYFQVQRRKRILNNPRDDYDFEMIEDENALHGGNG
RSGRTQRRGGELYNAFAGESDEEEPLFSDEDDEPYRDRAPSEDRLRDTS
SDDRSLRHGDH

An exemplary kex (kex2 proteinase) polypeptide sequence of Candida albicans having GenBank Accession No. AF022372.1 is provided below:

(SEQ ID NO: 11)
MLPIKLLIFILGYLLSPTLQQYQQIPPRDYENKNYFLVELNTTNSQKPL
IDFISHYRGHYNFEHQLSSLDNHYVFSIDKSHPHNSFLGNHNSNEYNLM
KRQLGHEQDYDELISHVESIHLLPMKKLSKRIPVPIEMEDVVFDNRDDT
GSDNHEATDEAHQKLIEIAKKLDIHDPEFTTQWHLINLKYPGHDVNVTG
LWLEDILGQGIVTALVDDGVDAESDDIKQNFNSEGSWDFNNKGKSPLPR
LFDDYHGTRCAGEIAAVKNDVCGIGVAWKSQVSGIRILSGPITSSDEAE
AMVYGLDTNDIYSCSWGPTDNGKVLSEPDVIVKKAMIKGIQEGRDKKGA
IYVFASGNGGRFGDSCNFDGYTNSIYSITVGAIDYKGLHPQYSEACSAV
MVVTYSSGSGEHIHTTDIKKKCSATHGGTSAAAPLASGIYSLILSANPN
LTWRDVQYISVLSATPINEEDGNYQTTALNRKYSHKYGYGKTDAYKMVH
FAKTWVNVKPQAWYYSDIIEVNQTITTTPEQKAPSKRDSPQKIIHSSVN
VSEKDLKIMNVERVEHITVKVNIDSTYRGRVGMRIISPTGVISDLATFR
VNDASTRGFQNWTFMSVAHWGETGIGEWKVEVFVDDSKGDQVEINFKDW
QFRIFGESIDGDKAEVYDITKDYAAIRRELLEKEKQNSKSTTTTSSTTT
ATTTSGGEGDQKTTTSAENKESTTKVDNSASITTSQTASLTSSNEQHQP
TESNSDSDSDTDDENKQEGEEDNDNDNDNGNKKANSDNTGFYLMSIAVV
GFIAVLLVMKFHKTPGSGRRRRRRDGYEFDIIPGEDYSDSDDDEDDSDT
RRADDDSFDLGHRNDQRVVSASQQQRQYDRQQDEARDRLFDDFNAESLP
DYENDMFKIGDEEEEEEEEEEGQQSAKAPSNSEGNSGTSTKK

An exemplary kex polypeptide sequence of Cryptococcus neoformans (JEC21) having NCBI Accession No. XM_572303.1 is provided below:

(SEQ ID NO: 12)
MRTLLSLWGILLALIVPPSLALQRPQPRSYDTHAYYALELDPSISPAAA
LQLSKSLGVELVERIGELDGHWLVRTEGWTPEHASITKRSVSHDPILKR
WEALPSSLGKKSLTPLSLKQRAKRHKSYSPRSRHSRDDRTELLYAQNEL
HLADPMLDQQWHLINTQMKDIELNVTGLWGRGITGEGVHVVIIDDGLDV
ESKDLKDNFFAEGSYDFNDHTELPIPRLKDDQHGTRCAGEIAAVPNDVC
GVGVAYDSKIAGVRILSAPISDADEAAALNYAYQLNDIYSCSWGPPDDG
RSMEAPDGLILKAMVNGVQKGRDGKGSVFVFAAGNGGGSDDQCNFDGYT
NSIFSVTVGAVDRKGLHPYYSEMCAAMMVVAPSSGSGDHIHTTDVGKDK
CSHSHGGTSAAAPLAVGVFALALSVRPDLTWRDIQHLAVRHAVFFNPDD
PAWELTAAGRHFSYKYGYGKLDAGLFVEAAEKWQLVKPQTWYDSPSVYL
PTTSPADVTRRQDEAADGPTSSDEETSNPPPVVEPSGSFITEDGVISTY
EVTQSMLFDANFERLEHVTVRVWIDHQRRGDVEVELTSPNGVVSVLCRQ
RRFDNADSGFPGWKFMSLKHWDENPVGTWTIKVKDQVNPDKTGRFVAWS
LQLWGESVDPALAKLWAPAEEGQPDEEQTGSNPSTTVSQKPKPTALLPG
DHGEASGEATQPGLGSATAHPQPTSTTGDAGNVAEPTGPTDADADEGFF
SGISNLASSSTWLAGAGAIIILSGAAIGAFFFIRARRQKRNLFGLSNNG
QGARGAYEPVDDVQMSLLERGRRKFGKSKSESQGTKDLYDAFGDGPSDE
EEEDLDERTALRYHDGFLEDDEPNEVGPKTEYKDEPESEPETFKDGEET
VGTKDKGKGKGPSEGESGSGSSSSWQDAADEEARV

By “agent” is meant a peptide, nucleic acid molecule, or small compound.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

The term “antibody,” as used herein, refers to an immunoglobulin molecule which specifically binds with an antigen. Methods of preparing antibodies are well known to those of ordinary skill in the science of immunology. Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. Antibodies also include dimers that may be naturally occurring or constructed from single chain antibodies or antibody fragments. The antibodies of the described embodiments may exist in a variety of forms including, for example, polyclonal antibodies, monoclonal antibodies, Fv, Fab and F(ab′)₂, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, scFv antibodies, single-domain antibodies, such as camelid antibodies (nanobodies), (Riechmann, 1999, Journal of Immunological Methods, 231:25-38), composed of either a V_{L} or a V_{H} domain which exhibit sufficient affinity for the target, and multispecific antibodies formed from antibody fragments. The antibody fragment also includes a human antibody or a humanized antibody or a portion of a human antibody or a humanized antibody.

Antibodies can be made by any of the methods known in the art utilizing a polypeptide or peptide (e.g., a kex peptide as described herein), or immunogenic peptide fragments thereof, as an immunogen. One method of obtaining antibodies is to immunize suitable host animals with an immunogen and to follow standard procedures for polyclonal or monoclonal antibody production. The immunogen facilitates the presentation of the immunogenic fragments on the cell surface. Immunization of a suitable host can be carried out in a number of ways. Nucleic acid sequences encoding a polypeptide as described herein, or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding the polypeptide, or immunogenic fragments thereof, can be expressed in cells in vitro, followed by isolation of the polypeptide and administration of the polypeptide to a suitable host in which antibodies are raised.

Alternatively, antibodies against the polypeptide may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be isolated, or isolated and purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. To produce large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition (e.g., Pristane).

By “anti-kexin antibody,” “anti-kex antibody,” or “anti-kex1 antibody” is meant an antibody or an antigen binding fragment thereof that selectively binds to a kexin polypeptide or a bindable fragment or portion thereof, or to a kex (kex1) peptide, including, for example, a kex (kex1) peptide of a fungal pathogen, such as Candida albicans, Pneumocystis hominis, Pneumocystis jirovecii (aka carinii), Aspergillus fumigatus, Cryptococcus neoformans, Histoplasma capsulatum, Coccidioides immitis, Mucor circinelloides, Rhizopus oryzae (also called Rhizopus arrhizus), Rhizopus delemar, and Candida auris, as described herein. In various embodiments, anti-kex (kex1) antibody or anti-kexin antibody specifically binds a binding site of a kexin protein or kex peptide, including a recombinant or recombinantly produced kex (kex1) peptide. In specific embodiments, the anti-kex (kex1) antibody specifically binds a binding site of a kexin protein or kex peptide of a fungal pathogen, for example, one or more of a Candida (e.g., Candida aurus or Candida albicans), Pneumocystis, Aspergillus, Cryptococcus, Histoplasma, Coccidioides, Mucor, and Rhizopus fungal pathogen.

An “antiserum” refers to blood serum that contains one or more antibodies directed against a specific antigen. Antiserum containing antibodies may be obtained from the blood or serum of an animal (a mammal), including a human, a rodent, or a non-human primate, that has been immunized or inoculated with an immunogen (or an antigen material) either by injection, typically into the bloodstream or tissues, or by infection. In an embodiment, an antiserum containing antibodies that cross-react with (bind to) kex peptides produced or expressed by Histoplasma capsulatum, Coccidioides immitis, Mucor circinelloides, Rhizopus oryzae (also called Rhizopus arrhizus), Rhizopus delemar, and Candida auris, including a recombinant or recombinantly produced kex (kex1) peptide, is generated or elicited by immunizing or innoculating an animal (recipient animal) with pan-fungal peptide 2 of SEQ ID NO: 7 (FIGS. 1A and 1B). In an embodiment, an antiserum containing antibodies that cross-react with (bind to) the kex peptides produced or expressed by other fungal pathogens is generated or elicited by immunizing or innoculating an animal (recipient animal) with a kex peptide immunogenic antigen produced by one or more of the following fungal pathogens: Histoplasma capsulatum, Coccidioides immitis, Mucor circinelloides, Rhizopus oryzae (also called Rhizopus arrhizus), Rhizopus delemar, and Candida auris. In an embodiment, the animal (a mammal), including a human or a non-human primate, may be immunized or inoculated with the blood or serum of an organism or individual whose immune system has been stimulated to generate an immune response (e.g., antibody production) by infection or natural contact with an antigenic material or immunogen. In an embodiment, an antiserum contains anti-kex peptide antibodies, e.g., polyclonal antibodies or populations of monoclonal antibodies, generated or produced by an immunized, inoculated, or exposed donor subject against a kex peptide immunogen, or a polynucleotide encoding the kex peptide immunogen, derived from a fungal pathogen. In embodiments, the kex peptide immunogen or the kex peptide immunogenic antigen is selected from pan-fungal peptide 2 of SEQ ID NO: 7, or is selected from one or more of SEQ ID NOs: 2-6 corresponding to fungal pathogens Histoplasma capsulatum, Coccidioides immitis, Mucor circinelloides, Rhizopus oryzae (also called Rhizopus arrhizus), Rhizopus delemar, and Candida auris, respectively.

Such antiserum, isolated (and/or purified) from the donor subject can be used to immunize (i.e., administer to) another (unrelated) subject to provide immunity (acquired immunity) against infection or disease caused by or associated with another fungal pathogen that produces a kex peptide that is also targeted and recognized by cross-reactive anti-kex peptide antibodies in the antiserum. The fungal pathogens may include one or more of Pneumocystis species (spp.), Candida spp, or Candida albicans, Aspergillus spp. or Aspergillus fumigatus, or Cryptococcus spp, or Cryptococcus neoformans, Histoplasma capsulatum, Coccidioides immitis, Mucor circinelloides, Rhizopus oryzae (also called Rhizopus arrhizus), Rhizopus delemar, and Candida auris. In this way, a subject who receives the antiserum, i.e., anti-kex peptide antibodies in the antiserum, is treated or protected against infection and/or disease caused by more than one fungal pathogen. Such antiserum-derived immunoprotection against multiple fungal pathogens constitutes an acquired or passive immunity obtained by the recipient subject and imparted from the donor subject's isolated antiserum. As will be appreciated by one skilled in the art, blood serum is the amber-colored, protein-rich liquid component of blood that separates from the clot when blood coagulates. The serum component containing one or more antibodies (cross-protective antibodies) is termed “antiserum.” In an embodiment, the antiserum is an isolated antiserum, e.g., isolated from a donor subject. In an embodiment, an isolated antiserum may be processed by methods known and used by one skilled in the art, such as dilution, concentration (e.g., via filtration or centrifugation or both), chromatography, purification to remove ions or extraneous protein, and the like, prior to its use as a treatment or protective therapeutic as described herein. In an embodiment, an isolated antiserum may be further purified after isolation. In an embodiment, an isolated antiserum is not further processed or purified. In an embodiment, antibodies, e.g., anti-kex peptide antigen antibodies, or antigen-binding fragments thereof, contained in an isolated antiserum may be further isolated by methods practiced by those having skill in the art, such as, without limitation, by affinity chromatography, size exclusion chromatography, immunoprecipitation, dialysis, HPLC chromatography, etc.

By “biological sample” is meant any liquid, cell, or tissue obtained from a subject. In some embodiments, the biological sample is blood, serum, plasma, cerebrospinal fluid, bronchoalveolar lavage, sputum, tears, saliva, urine, semen, feces, etc.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of an analyte that is detected or that is to be detected.

By “disease” is meant any condition, dysfunction, or disorder that damages or interferes with the normal function of a cell, tissue, or organ. In various embodiments, the disease is one that is caused by or associated with infection by a fungal pathogen, for example, Pneumocystis species (spp.), Candida spp, or Candida albicans, Aspergillus spp. or Aspergillus fumigatus, or Cryptococcus spp, or Cryptococcus neoformans, Histoplasma capsulatum, Coccidioides immitis, Mucor circinelloides, Rhizopus oryzae (also called Rhizopus arrhizus), Rhizopus delemar, and Candida auris. By way of example, diseases associated with Candida fungal pathogens, which typically reside in the intestinal tract and mucous membranes and can cause thrush, infections and invasive candidiasis upon systemic infection, especially in those in poor health or with weak immune systems; Cryptococcus, which can infect the lungs and cause pneumonia-like illness, and the brain and cause meningitis; Histoplasma, a fungus found in soil, particularly around areas where large amounts of bird or bat droppings accumulate, can cause histoplasmosis, resulting in (mild) flu-like symptoms of fever, chills, headache, cough and fatigue; Coccidioides, another soil fungus, which causes coddidioidomycosis or valley fever, results in infection with symptoms including fatigue, cough, fever, shortness of breath, headache, muscle aches, and rash, Mucor (e.g., Mucor circinelloides), found in soil, dung and root vegetables, is an emerging pathogen that can cause cutaneous infection of animals and humans; and Rhizopus, which are saprobic fungi, can cause disease, e.g., zygomycosis, in plants and animals, in particular, in burn victims, individuals suffering from severe malnutrition, patients with diabetic ketoacidosis, or immunocompromised individuals, such as those having HIV/AIDS or cancer, via invasion of blood vessels and progression to other areas of the body, including the brain and the lungs. In other embodiments, the disease is a pulmonary (lung) disease or a brain disease, e.g., meningitis. Non-limiting examples of pulmonary diseases include Chronic Obstructive Pulmonary Disease (COPD), which is a progressive lung disease that includes emphysema, chronic bronchitis, refractory (non-reversible) asthma, pneumonia (e.g., Pneumocystis pneumonia) and some forms of bronchiectasis.

By “effective amount” is meant the amount of an agent or therapeutic agent required to ameliorate the symptoms of a disease relative to an untreated patient. The effective amount of the agent, such as an active compound(s), an immunogenic kex peptide antigen, or an antiserum produced by immunization of a subject with an immunogenic kex peptide antigen, used to practice the methods as described herein for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. An immunologically effective amount of an immunogenic kex peptide antigen used to generate immunoreactive, anti-kex peptide antibodies in a subject following immunization or administration, or an isolated antiserum as described herein, is an amount required to treat a disease or the symptoms thereof associated with or caused by infection by one or more of the fungal pathogens described herein. By way of example, an effective amount of an immunogenic kex peptide antigen used to generate immunoreactive, anti-kex peptide antibodies in a subject following immunization or administration, or an isolated antiserum, may be determined by measuring the amount or titer of antibodies directed against the desired immunogen present in the serum by methods known and practiced in the art. The range of typical dosages for passive immunotherapy (i.e., the administration of antiserum containing antibodies) includes about 0.3 mg to about 100 mg/kg of total body weight. Following passive immunotherapy, treatment efficacy is typically conducted, as individual patients respond differently to therapies. Adjustment of the dosage may be modified as needed. Treatment regimens can be determined by methods known and practiced by those having skill in the art. In one embodiment, the amount is sufficient to induce an immune response.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. A fragment optimally maintains the activity and/or function of the polypeptide or polynucleotide from which it is derived. In embodiments, the fragment or portion maintains at least about 20%, 25%, 30%, 35%, 40%, 45%, 50%, 55%, 60%, 65%, 70%, 75%, 80%, 85%, 90%, 95%, 96%, 97%, 98%, 99%, or greater activity and/or function of the polypeptide or polynucleotide from which it is derived.

By “genetic vaccine” is meant an immunogenic composition comprising a polynucleotide encoding an antigen.

By “immune response” is meant is any activity of the immune system that is generated against an antigen (immunogenic antigen), such as a kex peptide antigen as described herein. In some embodiments, the immune response is an innate or an adoptive immune response that protects a subject from infection with a pathogen (e.g., fungal pathogen) or treats a pathogen infection. In some embodiments, an immune response involves the generation of antibodies against an antigen.

The term “immunocompromised” refers to a subject having a weakened or impaired immune system and/or associated immune response to a pathogen, pathogenic antigen, disease, etc. A subject may be immunocompromised as a consequence of taking immunosuppressive drugs, or by being afflicted with a disease or pathology that affects the subject's immune system, such as certain congenital diseases. The term “immunosuppressed” refers to a subject whose immune system and associated immune response to pathogens, pathogenic antigens, disease, etc. is partially or completely suppressed, for example, by a reduction in the activity or efficiency in the immune system. Immunosuppression of a subject's immune system or immune response may occur naturally due to a disease or disorder in the subject or may be induced in the subject by the administration of immunosuppressive agents, drugs, e.g., anti-cancer drugs, compounds, small molecule agents, and the like. In some cases, a subject who is immunosuppressed or is undergoing immunosuppression, or who has a weakened immune system due to a disease or condition (e.g., chemotherapy or an immune deficiency disease) is said to be immunocompromised.

By “immunogenic composition” is meant a composition comprising an antigen or immunogen, or immunogenic antigen, or a polynucleotide encoding the antigen or immunogen, wherein the composition elicits, produces, or generates an immune response in a subject immunized with or administered the composition.

The terms “isolated,” “purified,” or “biologically pure” refer to material that is free to varying degrees from components which normally accompany it as found in its native state or environment. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide as described herein is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” or “isolated peptide” is meant a polypeptide or peptide that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide or peptide as described herein. An isolated polypeptide or peptide as described herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, “obtaining” as in “obtaining an agent” includes synthesizing, purchasing, or otherwise acquiring the agent.

By “opportunistic infection” is meant an infection caused by pathogens such as fungal pathogens, bacteria, viruses, protozoa, or parasites that take advantage of an opportunity to infect a subject (host) that is not normally available, for example, a host having a weakened immune system, an immunocompromised host, an immunosuppressed host, a host with altered microbiota or microflora, or a host having protective integumentary barriers that have been damaged or breached. In an embodiment, an opportunistic infection is caused by one or more fungal pathogens as described herein.

By “reduces” or “diminishes’ is meant a negative alteration of at least 10%, 25%, 50%, 75%, or 100%.

By “reference” is meant a standard or control condition. A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody or antigen binding fragment thereof that recognizes and binds a polypeptide or peptide, but which does not substantially recognize and bind other molecules in a sample, for example, a biological sample, which naturally includes a polypeptide or peptide as described herein. Cross-reactive binding includes specific binding (e.g., by an antibody or an antigen binding fragment thereof) to an original polypeptide or peptide antigen/immunogen as well as binding to a polypeptide or peptide other than the original antigen/immunogen. In a nonlimiting embodiment, the original and cross-reactive polypeptide or peptide antigen/immunogen may have or contain structural or conformational similarities, epitopes, ionic charge, and the like.

Nucleic acid molecules useful in generating a recombinant immunogen or a vaccine include any nucleic acid molecule that encodes a polypeptide or a peptide fragment thereof, such as a kex peptide described herein. Such nucleic acid molecules need not be 100% identical with an endogenous nucleic acid sequence, but will typically exhibit substantial identity to an endogenous sequence. Polynucleotides having “substantial identity” to an endogenous sequence are typically capable of hybridizing with at least one strand of a double-stranded nucleic acid molecule. Nucleic acid molecules may include any nucleic acid molecule that encodes a polypeptide or a peptide fragment thereof. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a non-human primate, or a murine, bovine, equine, canine, ovine, or feline mammal. In an embodiment, the subject is a human. In an embodiment, a subject is a human patient who is undergoing treatment for disease or disease symptoms caused by or associated with infection by one or more pathogenic fungi, such as, for example, Pneumocystis, Aspergillus, Candida, or Cryptococcus, or Histoplasma capsulatum, Coccidioides immitis, Mucor circinelloides, Rhizopus oryzae (also called Rhizopus arrhizus), Rhizopus delemar, and Candida auris. In an embodiment as subject is a human patient who is at risk of infection (e.g., opportunistic infection) or disease caused by one or more pathogenic fungi, such as Pneumocystis, Aspergillus, Candida, or Cryptococcus, or Histoplasma capsulatum, Coccidioides immitis, Mucor circinelloides, Rhizopus oryzae (also called Rhizopus arrhizus), Rhizopus delemar, and Candida auris. In an embodiment, a subject is a mammalian (e.g., a human; a non-human primate) donor subject from whom antiserum containing anti-fungal kex peptide antibodies is obtained or isolated. In an embodiment, a subject is a mammalian (e.g., a human; a non-human primate) recipient subject who receives an isolated antiserum composed of anti-fungal kex peptide antibodies and acquires protective immunity (and treatment) against multiple fungal pathogens.

As used herein, a “vector” refers to a nucleic acid (polynucleotide) molecule into which foreign nucleic acid can be inserted without disrupting the ability of the vector to replicate in and/or integrate into a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that contains the necessary regulatory sequences to allow transcription and translation of inserted gene or genes in a host cell. One skilled in the art will recognize that depending on the expression vector used, additional nucleotides may need to be added to the 5′ end of the foreign nucleic acid to be inserted into the vector to maintain the proper reading frame.

By “vaccine” is meant a preparation of immunogenic material (e.g., protein or nucleic acid; vaccine) capable of stimulating (eliciting) an immune response, administered to a subject to treat a disease, condition, or pathology, or to protect against or prevent a disease, condition, or pathology, and/or the symptoms thereof, such as an infectious disease (caused by a fungal pathogen, for example). The immunogenic material may include, for example, attenuated or killed microorganisms (such as attenuated viruses), or antigenic proteins or peptides, e.g., kex peptide antigens, or DNA derived from such microorganisms. Vaccines may elicit a prophylactic (protective or preventative) immune response in the subject; they may also elicit a therapeutic response or immune response in a subject. As mentioned above, methods of vaccine administration vary according to the vaccine, and can include routes or means, such as inoculation (intravenous or subcutaneous injection), ingestion, inhalation, or other forms of administration. Inoculations can be delivered by any number of routes, including parenteral, such as intravenous, subcutaneous or intramuscular. Vaccines may also be administered with an adjuvant to boost the immune response.

Ranges provided herein are understood to be shorthand for all the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing, abating, diminishing, alleviating, eliminating, or ameliorating a disease, disorder and/or symptoms associated therewith. It will be appreciated that, although not precluded, treating a disease, disorder and/or symptoms associated therewith does not require that the disease, disorder, condition or symptoms associated therewith be eliminated.

As used herein, a therapeutic that “prevents” a disorder or condition refers to a compound or material that, in a statistical sample, reduces, protects against, or stops the occurrence of the disease, disorder or condition in the treated subject or sample relative to an untreated control subject or sample, or delays the onset or reduces the severity of one or more symptoms of the disease, disorder, or condition relative to an untreated control subject or sample. In an embodiment, a protective or preventive therapeutic is an antibody or an antigen binding fragment thereof. In an embodiment, the antibody is generated against a kex peptide of a fungal pathogen as described herein. In a particular embodiment, a protective or preventive therapeutic is an isolated antiserum containing anti-kex peptide antibodies or antigen binding fragments thereof generated against a kex peptide of a fungal pathogen as described herein.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

### BRIEF DESCRIPTION OF THE DRAWINGS

Exemplary embodiments as described herein are provided below as drawings and figures related to the described aspects and embodiments in their various and nonlimiting aspects.

FIGS. 1A-1C provide Western blots and a graph showing that immunization of mice with the pan-fungal peptide 2 generated antifungal cross-reactive antibodies. The pan-fungal peptide 2 sequence (SEQ ID NO: 7) was generated based on a consensus sequence with homology to kex1 regions of Pneumocystis (PC.KEX1), Aspergillus (AF.KEX1), Candida (CA.KEX1), and Cryptococcus (CN.KEX1) by amino acid type (Clustal Omega), as described in U.S. Publication No. 2022/0184190, and in E. Rayens et al., 2022, PNAS Nexus, 1(5):pgac248 (doi: 10.1093/pnasnexus/pgac248), the contents of which are incorporated by reference herein. In FIGS. 1A-1C, pan-fungal peptide 2 is termed “NXT-2.” FIGS. 1A and 1B show the percent identity of each of the KEX1 peptide sequences compared to that of pan-fungal peptide 2 (“NXT-2”), and the western blots developed using plasma from non-human primates (NHPs) immunized with pan-fungal peptide 2+alum or PBS+alum. Antibodies directed against pan-fungal peptide 2 (“NXT-2”) bound to Histoplasma capsulatum KEX1 (HC.KEX2; FIG. 1A, lane 3 of Blot), Coccidioides immitis KEX1 (CI.KEX1; FIG. 1A, lane 4 of Blot), Mucor circinelloides KEX1 (MC.KEX1; FIG. 1A, lane 5 of Blot), Candida auris KEX1 (CAu.KEX1; FIG. 1B, lane 3 of Blot), and C. neoformans KEX1 (CN.KEX1; FIG. 1B, lane 4 of Blot). FIG. 1C presents a graph showing the correlation of % identity and the % western blot saturation quantified by ImageJ relative to pan-fungal peptide 2 (NXT-2), (P=0.0009).

FIGS. 2A-2C show SDS-PAGE gel and western blot analysis of the immunogenicity of recombiant Candida auris kex peptide (CAu.KEX1). The CAu.KEX1 recombinant protein was purified from E. coli as described in Example 8, Methods. The purified peptide was electrophoresced on SDS-PAGE gels stained with Coomassie Blue (FIG. 2A) or transferred to nitrocellulose membranes for western blotting with plasma from CAu.KEX1 immunized mice (FIG. 2B) or an immunized non-human primate (NHP), (FIG. 2C). The recombinant CAu.KEX1 protein was shown to be immunogenic and to induce robust antibody production.

FIG. 3 shows bar graphs illustrating that anti-CAu.KEX1 peptide antibodies promote opsonophagocytic killing of C. auris fungal cells. Plasma obtained from mice immunized with recombinant CAu.KEX1 peptide contains antibodies that significantly enhanced the killing of C. auris cells by macrophages compared to plasma obtained from sham immunized mice. The data were analyzed using Mann-Whitney test and p=0.03.

### DETAILED DESCRIPTION

Featured and described herein are kex (kex1) peptide immunogens or immunogenic antigens. The kex peptides are produced, e.g., recombinantly produced, expressed, or derived from a pathogenic fungal organism as described herein. Also provided are compositions and pharmaceutically acceptable compositions containing such kex peptide immunogens and immunogenic antigens, and methods of use. Methods of treating or protecting against disease, severity or disease, pathologies, and/or symptoms of disease or pathology caused by or associated with infection (e.g., opportunistic infection) by one or more fungal pathogens are provided and described herein. In some cases, the fungal pathogens are invasive fungal organisms of different etiologies.

By way of specific example, disease-causing fungal pathogens include those in the genera Candida, which typically reside in the intestinal tract and mucous membranes and can cause thrush, infections and invasive candidiasis upon systemic infection, especially in those in poor health or with weak immune systems; Cryptococcus, which can infect the lungs and cause pneumonia-like illness, as well as the brain and cause meningitis; Histoplasma, a fungus found in soil, particularly around areas where large amounts of bird or bat droppings accumulate, can cause histoplasmosis, resulting in (mild) flu-like symptoms of fever, chills, headache, cough and fatigue; Coccidioides, another soil fungus, which causes coddidioidomycosis or valley fever, results in infection with symptoms including fatigue, cough, fever, shortness of breath, headache, muscle aches, and rash, Mucor (e.g., Mucor circinelloides), found in soil, dung and root vegetables, is an emerging pathogen that can cause cutaneous infection of animals and humans; and Rhizopus, which are saprobic fungi, can cause disease, e.g., zygomycosis, in plants and animals, in particular, in burn victims, individuals suffering from severe malnutrition, patients with diabetic ketoacidosis, or immunocompromised individuals, such as those having HIV/AIDS or cancer, via invasion of blood vessels and progression to other areas of the body, including the brain and the lungs. Other disease-causing fungal pathogens include Pneumocystis, which colonizes lung tissue and causes severe pneumonia after infection, and Aspergillus, a common mold, which causes aspergillosis, allergic reactions, lung infections and other health problems.

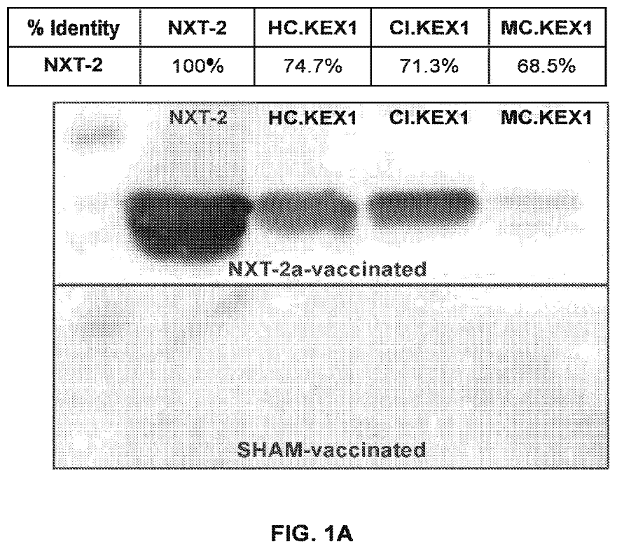

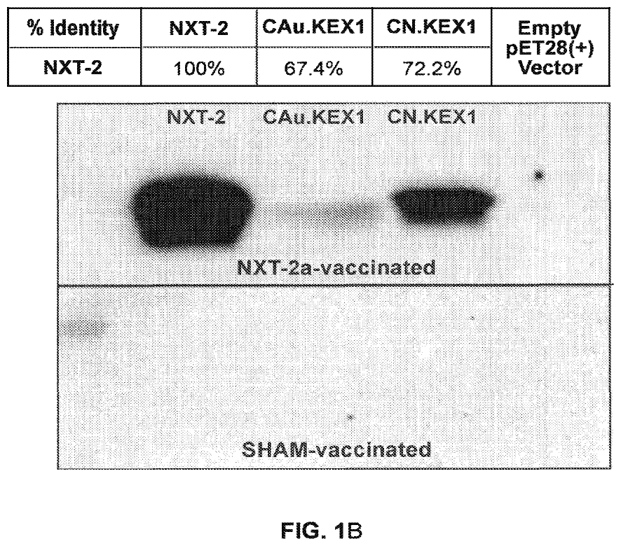

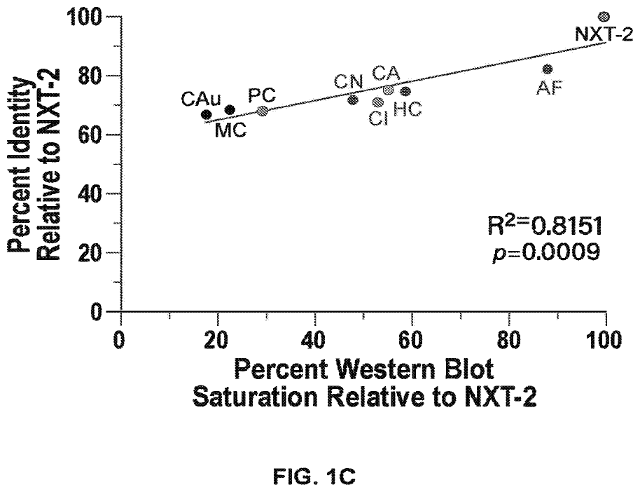

The described aspects and embodiments are based, at least in part, on the discovery that mammalian subjects, immunized with a non-naturally occurring, recombinantly produced, kex peptide (referred to herein as pan-fungal peptide 2 having an amino acid sequence as set forth in SEQ ID NO: 7) generated antibodies that specifically bound to and were cross-reactive with recombinant kex (kex1) peptides of a number of other distinct and different fungal pathogens, namely, Histoplasma capsulatum, Coccidioides immitis, Mucor circinelloides, Rhizopus oryzae (also called Rhizopus arrhizus), Rhizopus delemar, or Candida auris, having amino acid sequences as set forth in SEQ ID NOs: 1-6, respectively, as described herein, as well as to Aspergillus (Aspergillus fumigatus), Cryptococcus. Pneumocystis pneumoniae. (FIGS. 1A and 1B). The cross-reactivity of the anti-pan-fungal peptide 2 antibodies with the recombinant kex peptide antigens of other, distinct fungal pathogens indicates additional pathogens to which immunization with recombinant kex peptide antigen 2 may provide immunologic and therapeutic protection and treatment.

The several fungal-derived, recombinant kex peptide antigens as described herein are useful as immunogens, immunogenic antigens, and immunogenic antigens in immunogenic vaccines for generating anti-kex peptide antibodies and antisera containing anti-kex peptide antibodies following immunization of a subject with the immunogenic kex peptide antigens or vaccines containing these peptide antigens. In an embodiment, kex peptides as described herein, are derived from Histoplasma capsulatum, Coccidioides immitis, Mucor circinelloides, Rhizopus oryzae (also called Rhizopus arrhizus), Rhizopus delemar, or Candida auris (e.g., SEQ ID NOs: 1-6), and are useful as immunogens, immunogenic antigens, and immunogenic antigens in immunogenic vaccines. The anti-kex peptide antibodies and antisera containing the antibodies generated from administration or immunization of animals, including non-human primates and humans, with the kex peptide immunogens or immunogenic vaccines composed of kex peptide immunogens derived from Histoplasma capsulatum, Coccidioides immitis, Mucor circinelloides, Rhizopus oryzae (also called Rhizopus arrhizus), Rhizopus delemar, or Candida auris can be utilized in the treatment and/or protection of a subject against disesase, disease severity, and/or disease symptoms caused by the fungal pathogens themselves or one or more different fungal infections, due to the cross-reactivity of the anti-kex peptide antibodies resulting from the immune response elicited in the animal following administration or immunization. Thus the antibodies or antisera generated in an animal immunized with an immunogenic kex peptide antigen or immunogenic vaccine containing the kex peptide antigen of one fungal type or species can cross-react with kex peptides of one or more other fungal pathogens as described above. The cross-reactive nature of the anti-kex peptide antibodies generated against a given kex peptide immunogen or vaccine as described herein serves to treat and/or protect a subject from disease caused by more than one kex-peptide-producting fungal pathogen.

Antibody-containing antiserum generated in response to administration of the non-naturally occurring, recombinantly produced pan-fungal kex peptide 2, or an encoding polynucleotide, can also serve as a treatment or protection from disease caused by or associated with fungal pathogens Histoplasma capsulatum, Coccidioides immitis, Mucor circinelloides, Rhizopus oryzae (also called Rhizopus arrhizus), Rhizopus delemar, or Candida auris, as antibodies in the antiserum were found to cross-react with the kex (kex1) peptides of these discrete and different fungal pathogens. Thus, such antibodies generated in one animal subject can provide immunity (e.g., treatment or protection) against disease or disease symptoms caused by multiple other fungal pathogens when administered to another or unrelated subject (i.e., a recipient subject) via a suitable mode and route of administration. It will be appreciated by the skilled practitioner that, as used herein, a subject from whom an antiserum is obtained or isolated is a “donor subject,” and a subject to whom the isolated antiserum is administered or provided is a “recipient subject.” In embodiments, a subject is a mammal, particularly a human being or a non-human primate. A recipient subject may be a patient or an individual in need of treatment for or protection from disease or disease symptoms caused by one or more fungal pathogens.

The production of such immunologically cross reactive antisera (and antibodies therein) produced in subjects (e.g., donor subjects) immunized with, or exposed to, pan-fungal kex peptide 2, or a polynucleotide encoding the pan-fungal kex peptide 2, that reacted with the kex peptides of multiple fungal pathogens, such as the fungal pathogens Histoplasma capsulatum, Coccidioides immitis, Mucor circinelloides, Rhizopus oryzae (also called Rhizopus arrhizus), Rhizopus delemar, or Candida auris as described herein, was surprising and unexpected, particularly in view of the low amount of amino acid sequence identity (e.g., about 48%-70% variability) that may exist among the kex peptides of different fungal pathogens.

Also embraced herein is an immunogenic composition comprising a pan-fungal peptide, or a polynucleotide encoding the pan-fungal peptide, that elicits a potent immune response in a subject following administration of the composition and the production of antiserum in the subject that contains one or more antibodies or antigen binding fragments thereof that reacts not only with the immunizing antigen, but also with the distinct and different fungal pathogens Histoplasma capsulatum, Coccidioides immitis, Mucor circinelloides, Rhizopus oryzae (also called Rhizopus arrhizus), Rhizopus delemar, or Candida auris.

One benefit of the described methods is the provision of treatment, protection, or prevention of infection by and disease associated with several different fungal pathogens using a composition comprising a non-naturally occurring, recombinant kex peptide immunogen or an immunogenic vaccine containing the recombinant kex peptide immunogen. By way of example, the non-naturally occurring, recombinant kex peptide immunogen or an immunogenic vaccine containing the recombinant kex peptide immunogen may be from one or more of Histoplasma capsulatum, Coccidioides immitis, Mucor circinelloides, Rhizopus oryzae (also called Rhizopus arrhizus), Rhizopus delemar, or Candida auris fungal pathogens. An antiserum (isolated antiserum) generated in response to administration of the non-naturally occurring kex peptide immunogen, or a polynucleotide encoding the kex peptide, or a vaccine containing the kex peptide immunogen can contain anti-kex peptide antibodies that cross-react with the kex peptide of another, different fungal pathogen and protects against multiple fungal organisms and treats, protects against, or prevents diseases and symptoms thereof, pulmonary disease and poor pulmonary performance, associated with infection (and colonization) by the different fungal pathogens, e.g., one or more fungal pathogens. In an embodiment, the different fungal pathogens include the Histoplasma capsulatum, Coccidioides immitis, Mucor circinelloides, Rhizopus oryzae (also called Rhizopus arrhizus), Rhizopus delemar, or Candida auris fungal pathogens. In one embodiment, a composition is provided that comprises a non-naturally occurring, recombinantly produced kex peptide immunogen, or a polynucleotide encoding the kex peptide, that elicits the production of antibodies that specifically react with a kex peptide derived from one or more of Histoplasma capsulatum, Coccidioides immitis, Mucor circinelloides, Rhizopus oryzae (also called Rhizopus arrhizus), Rhizopus delemar, or Candida auris fungal pathogens, or with Pneumocystis (Pneumocystis jirovecii), Aspergillus (Aspergillus fumigatus), Candida (Candida albicans), or Cryptococcus (Cryptococcus neoformans). Accordingly, the antiserum is cross-protective (e.g., cross-reactive with kex peptides of multiple fungal types) and affords treatment and/or protection against disease or disease symptoms associated with infection by multiple fungal organisms when provided to another (e.g., unrelated) subject in need thereof. In an embodiment, the antiserum is an isolated antiserum. In an embodiment, the isolated antiserum is administered in a pharmaceutically acceptable composition.

The methods and compositions described herein offer economic, medical and practical benefits in the treatment, protection against, and prevention of fungal infection and disease, such as pulmonary disease, or types of brain infections, associated with infection and colonization by different types of fungal pathogens.

### Therapeutic Methods

The methods and compositions provided herein can be used to treat, protect against, or prevent diseases and/or symptoms thereof caused by fungal pathogens. In particular, the fungal pathogens include Histoplasma capsulatum, Coccidioides immitis, Mucor circinelloides, Rhizopus oryzae (also called Rhizopus arrhizus), Rhizopus delemar, or Candida auris. In other embodiments, the fungal pathogens include Pneumocystis, Aspergillus, Candida, and Cryptococcus, and, in particular, Pneumocystis hominis or jirovecii, Aspergillus fumigatus, Candida albicans, or Cryptococcus neoformans. The methods and compositions provided herein can provide immune protection in a subject against disease caused by at least one, or more than one, of these fungal organisms. The methods and compositions provided herein can immunize a recipient subject to treat or protect against disease and/or the symptoms thereof associated with or caused by infection (or exposure to) at least one or more than one of these fungal organisms.

A non-naturally occurring, recombinant (or recombinantly produced), immunogenic kex peptide antigen of Histoplasma capsulatum, Coccidioides immitis, Mucor circinelloides, Rhizopus oryzae (also called Rhizopus arrhizus), Rhizopus delemar, or Candida auris, or a an immunogenic composition comprising the kex peptide, or a polynucleotide encoding the kex peptide can be administered to a subject to provide therapeutic and/or prophylactic immunity against the fungal pathogen from which the kex peptide is derived (e.g., the source fungal pathogen) and/or against other pathogenic fungal organisms that express a kex protein or peptide antigen against which antibodies (antiserum) generated in the immunized subject cross-react (and bind). In embodiments, an antiserum or isolated antiserum containing one or more antibodies generated against the non-naturally occurring, recombinant (or recombinantly produced), immunogenic kex peptide antigen of Histoplasma capsulatum, Coccidioides immitis, Mucor circinelloides, Rhizopus oryzae (also called Rhizopus arrhizus), Rhizopus delemar, or Candida auris, can be administered therapeutically and/or prophylactically to provide immunity against the source fungal pathogen and/or against other pathogenic fungal organisms that express a kex protein or peptide antigen against which antibodies (antiserum) generated in the immunized subject cross-react (and bind), for example, the Histoplasma capsulatum, Coccidioides immitis, Mucor circinelloides, Rhizopus oryzae (also called Rhizopus arrhizus), Rhizopus delemar, or Candida auris fungal organisms. The methods include administering an immunologically effective amount of an immunogenic composition comprising a non-naturally occurring, recombinant (or recombinantly produced), immunogenic kex peptide antigen, or the encoding polynucleotide thereof, of Histoplasma capsulatum, Coccidioides immitis, Mucor circinelloides, Rhizopus oryzae (also called Rhizopus arrhizus), Rhizopus delemar, or Candida auris to produce, elicit, or generate antibodies that react against the kex peptide antigens of the fungal organisms and/or that react against the kex peptide antigens of one or more of the other fungal organisms.

Also provided is an antiserum or isolated antiserum containing antibodies generated by immunizing a subject with the pan-fungal kex peptide 2 (SEQ ID NO: 7) antigen, a polynucleotide encoding the pan-fungal kex peptide 2, or an immunogenic composition comprising the peptide or polynucleotide encoding the pan-fungal peptide 2 as immunogenic antigen, wherein the antibodies of the antiserum bind to and cross-react with a kex peptide antigen of one or more fungal pathogens selected from Histoplasma capsulatum, Coccidioides immitis, Mucor circinelloides, Rhizopus oryzae (also called Rhizopus arrhizus), Rhizopus delemar, or Candida auris.

Also provided is an antiserum or isolated antiserum containing antibodies generated by immunizing a subject with the pan-fungal kex peptide 2 (SEQ ID NO: 7) antigen, a polynucleotide encoding the pan-fungal kex peptide 2, or an immunogenic composition comprising the peptide or polynucleotide encoding the pan-fungal peptide 2 as immunogenic antigen, which provides cross-protective therapeutic or protective immunity to a subject who is at risk of, susceptible to, or has disease or symptoms thereof associated with or related to infection by a fungal pathogen selected from Histoplasma capsulatum, Coccidioides immitis, Mucor circinelloides, Rhizopus oryzae (also called Rhizopus arrhizus), Rhizopus delemar, or Candida auris and combinations of these fungal pathogens.

Further provided is an antiserum or isolated antiserum containing antibodies generated by immunizing a subject with a recombinant, immunogenic kex peptide antigen of Candida auris, Histoplasma capsulatum, Coccidioides immitis, Mucor circinelloides, Rhizopus oryzae (also called Rhizopus arrhizus), Rhizopus delemar, a polynucleotide encoding the immunogenic kex peptide antigen, or an immunogenic composition comprising the immunogenic kex peptide antigen or polynucleotide encoding the immunogenic kex peptide antigen. Such an antiserum or isolated antiserum can be used to provide therapeutic or protective immunity to a subject who is at risk of, susceptible to, or has disease or symptoms thereof associated with or related to infection by a fungal pathogen selected from Histoplasma capsulatum, Coccidioides immitis, Mucor circinelloides, Rhizopus oryzae (also called Rhizopus arrhizus), Rhizopus delemar, or Candida auris and combinations of these fungal pathogens.

The above-described antisera, or isolated antisera (or immune sera or immune plasma) can be administered to an individual, alone, or in a physiologically acceptable carrier, excipient, or diluent. In an embodiment, the immunogenic composition containing an immunogenic kex peptide antigen of the specified fungal pathogens, a polynucleotide encoding the immunogenic kex peptide antigen, or antiserum (isolated antiserum) generated against the immunogenic kex peptide antigen of the specified fungal pathogens, is a pharmaceutically acceptable composition.

Provided and described herein are methods of treating or protecting against disease or symptoms thereof associated with or related to infection by, or exposure to, a fungal pathogen selected from Histoplasma capsulatum, Coccidioides immitis, Mucor circinelloides, Rhizopus oryzae (also called Rhizopus arrhizus), Rhizopus delemar, or Candida auris, or a combination thereof, in which the methods involve administering to a subject (e.g., a mammal, including a human, a non-human primate, or a rodent) a therapeutically effective amount of an immunogenic composition comprising a non-naturally occurring, recombinant, pan-fungal kex peptide 2 (SEQ ID NO: 7), a polynucleotide encoding the pan-fungal kex peptide 2, or an isolated antiserum generated in response to immunization with the non-naturally occurring, recombinant, pan-fungal kex peptide 2 to a recipient subject (e.g., a mammal such as a human patient) in need thereof. In an embodiment, the antiserum contains antibodies that specifically target the kex peptide of Histoplasma capsulatum, Coccidioides immitis, Mucor circinelloides, Rhizopus oryzae (also called Rhizopus arrhizus), Rhizopus delemar, or Candida auris, to neutralize the activity of kex proteinase of these fungal pathogens. In an embodiment, the isolated antiserum allows the recipient subject to achieve and passively acquire protective immunity against these different fungal pathogens. In embodiments, the subject is protected against or immune to the onset of, or the severity of, diseases and/or the symptoms thereof caused by or associated with infection by these fungal pathogens or exposure to these fungal pathogens.

In an embodiment, the methods include the step of administering to a recipient mammal a prophylactic, protective, or preventive amount of an immunogenic composition comprising a non-naturally occurring, recombinant, immunogenic kex peptide of one or more of SEQ ID NOs: 1-7, a polynucleotide encoding the immunogenic kex peptide, or an antiserum generated in response to the immunogenic kex peptide under conditions sufficient to treat, protect against, ameliorate, or prevent disease or disease severity, and/or the symptoms thereof, caused by or associated with one or more of Histoplasma capsulatum, Coccidioides immitis, Mucor circinelloides, Rhizopus oryzae (also called Rhizopus arrhizus), Rhizopus delemar, or Candida auris fungal pathogens. In an embodiment, the immunogenic composition is a pharmaceutically acceptable composition. In an embodiment, the recipient mammal is a human patient in need of treatment.

Treatment will be suitably administered to subjects, particularly humans, suffering from, having, susceptible to, or at risk for disease and/or disease symptoms caused by or associated with infection by one or more of Histoplasma capsulatum, Coccidioides immitis, Mucor circinelloides, Rhizopus oryzae (also called Rhizopus arrhizus), Rhizopus delemar, or Candida auris fungal pathogens. In embodiments, the fungal pathogens may also include Pneumocystis, Aspergillus, Cryptococcus, in particular, Pneumocystis hominis or jirovecii, Aspergillus fumigatus, Candida albicans, or Cryptococcus neoformans. Determination of those subjects who are “at risk” or “susceptible to” can be made by any objective or subjective determination by a diagnostic test or opinion of a subject or health care provider (e.g., genetic test, enzyme test or assay, or protein marker (such as levels of anti-kex antibodies, e.g., in serum, family history, and the like). The methods herein also include administering to the recipient subject in need thereof (including a subject identified as in need of such treatment or as being at risk of disease caused by fungal infection) an effective amount of an anti-fungal pathogen immunogenic composition comprising a non-naturally occurring, recombinant, immunogenic kex peptide of SEQ ID NO: 1-7, or a polynucleotide encoding the immunogenic kex peptide. Methods described herein also include administering to a recipient subject in need thereof, an antiserum generated in and obtained or isolated from a (donor) subject who has produced such an antiserum in response to immunization with or exposure to an immunogenic kex peptide of one or more of SEQ ID NOs: 1-7 as described herein. Identifying a subject in need of such treatment can involve the judgment of the recipient subject or a health care or medical professional and can be subjective (e.g., opinion) or objective (e.g., measurable by a test or diagnostic method). In an embodiment, the isolated antiserum is provided in a pharmaceutically acceptable composition.

In some aspects, methods of treating, protecting against, or preventing a fungal pathogen-associated disease, condition, and/or symptoms thereof, caused by or associated with one or more of Histoplasma capsulatum, Coccidioides immitis, Mucor circinelloides, Rhizopus oryzae (also called Rhizopus arrhizus), Rhizopus delemar, or Candida auris fungal pathogens in a subject are featured, in which the methods involve administering to a subject in need thereof an effective amount of an immunogenic composition comprising a non-naturally occurring, recombinant, immunogenic kex peptide, a polynucleotide encoding the immunogenic kex peptide, or an isolated antiserum generated in response to the immunogenic kex peptide, wherein the kex peptide is as set forth in one or more of SEQ ID NOs: 1-6; and wherein the antiserum is obtained from an individual who has produced an antibody immune response against the immunogenic kex peptide of one or more of SEQ ID NOs: 1-6, such that the subject is therapeutically and/or prophylactically treated against disease or symptoms thereof associated with infection by or exposure to one or more of Histoplasma capsulatum, Coccidioides immitis, Mucor circinelloides, Rhizopus oryzae (also called Rhizopus arrhizus), Rhizopus delemar, or Candida auris fungal pathogens. Provided in another aspect are methods of treating or protecting a patient against disease and/or the symptoms thereof associated with or caused by infection by one or more of the Histoplasma capsulatum, Coccidioides immitis, Mucor circinelloides, Rhizopus oryzae (also called Rhizopus arrhizus), Rhizopus delemar, or Candida auris fungal pathogens, wherein the patient is receiving or who has received immune suppressive drugs or medication and who, as a result of drug-induced immune system suppression, is susceptible to or may become susceptible to (or at risk of) infection by a pathogenic fungus, such as one or more of Histoplasma capsulatum, Coccidioides immitis, Mucor circinelloides, Rhizopus oryzae (also called Rhizopus arrhizus), Rhizopus delemar, or Candida auris fungal pathogens, either in or outside of a nosocomial environment. By way of example, such a patient may be preparing to undergo a transplant (a pre-transplant patient) or may have received a transplant (a post-transplant patient) and is administered one or more immunosuppressive drugs or medications (anti-rejection medications) and/or is otherwise treated with drugs to reduce the likelihood of rejection of the transplanted organ or tissue, thereby making the patient more vulnerable, susceptible to, or at risk of infection and/or disease caused by one or more of the Histoplasma capsulatum, Coccidioides immitis, Mucor circinelloides, Rhizopus oryzae (also called Rhizopus arrhizus), Rhizopus delemar, or Candida auris fungal pathogens. Patients having other types of diseases and conditions, such as rheumatoid arthritis or psoriasis, and the like, may also be administered medications having an immune suppressive effect to treat or manage their conditions and thus may suffer from, or be at risk of, disease associated with infection by one or more of these fungal pathogens. Non-limiting classes of immune suppressive drugs and medications include, for example, corticosteroids, such as prednisone (e.g., DELATSONE, ORASONE); budesonide (ENTOCORT EC), or prednisolone (MLLIPRED) calcineurin inhibitors, such as cyclosporine (NEORAL, SANDIMMUNE, SANGCYA); or tacrolimus (ASTAGRAF XL, ENVARSUS XR, PROGRAF); mTOR inhibitors, such as sirolimus (RAPAMUNE), everolimus (AFINITOR, ZORTRESS); Inosine Monophosphate Dehydrogenase (IMDH) inhibitors, such as azathioprine (AZASAN, IMURAN), leflunomide (ARAVA), mycophenolate (CELLCEPT, MYFORTIC); Biologics and monoclonal antibodies or monoclonal antibody-based antibodies or antigen binding fragments thereof, such as abatacept (ORENCIA); adalimumab (HUMIRA); anakinra (KINERET); certolizumab (CIMZIA); etanercept (ENBREL); golimumab (SIMPONI); infliximab (REMICADE); ixekizumab (TALTZ); natalizumab (TYSABRI); rituximab (RITIXAN); secukinumab (COSENTYX); tocilizumab (ACTEMRA); ustekinumab (STELARA); and vedolizumab (ENTYVIO). In an embodiment, the patient is to receive or has received a transplant of an organ selected from kidney, liver, heart, bone marrow, pancreas, lung, gall bladder, bladder, etc.

Immunogenic compositions comprising a non-naturally occurring, recombinant, immunogenic kex peptide antigen of any one of SEQ ID NOs: 1-6, a polynucleotide encoding the immunogenic kex peptide antigen, or antibodies directed against the immunogenic kex peptide antigens (or antiserum containing such antibodies) can be administered to the patient who is receiving transplant rejection medication, or other immune suppressive medication, in an effective amount to heighten the immune response against disease, pathology, or symptoms thereof, associated with infection by one or more of the Histoplasma capsulatum, Coccidioides immitis, Mucor circinelloides, Rhizopus oryzae (also called Rhizopus arrhizus), Rhizopus delemar, or Candida auris fungal pathogens in the immune suppressed patient. In an embodiment, the patient receiving immune suppressing drugs can be evaluated and monitored during treatment with immune suppressive drugs for the presence of antibodies (and antibody titers) against one or more of the fungal pathogens by employing the methods and kits as described herein.

Optionally, an immunogenic composition comprising a non-naturally occurring, recombinant, immunogenic kex peptide antigen of any one of SEQ ID NOs: 1-6, a polynucleotide encoding the immunogenic kex peptide antigen, or antibodies directed against the immunogenic kex peptide antigen (or antiserum containing such antibodies) may be administered to a subject in combination with one or more of any other treatment or therapy, e.g., anti-fungal therapies. For example, such administration may be carried out in combination with other antibodies or antibody cocktails with anti-fungal activity (including, for example, immune plasma), or in combination with one or more drugs, for examples, one or more drugs having anti-fungal activity (e.g., trimethoprim-sulfamethoxazole, azithromycin-sulfamethoxazole, clarithromycin-sulfamethoxazole, atovaquone, sulfadoxine-pyrimethamine, erythromycin-sulfisoxazole, PS-15, and dapsone-trimethoprim, as well as intravenous pentamidine and clindamycin-primaquine), to provide protective immunity in the recipient against one or more of the Histoplasma capsulatum, Coccidioides immitis, Mucor circinelloides, Rhizopus oryzae (also called Rhizopus arrhizus), Rhizopus delemar, or Candida auris fungal organisms. In an embodiment of any of the foregoing, the immunogenic composition is provided as a pharmaceutically acceptable composition, e.g., containing a pharmaceutically acceptable carrier, excipient, or diluent. In an embodiment of the foregoing, the immunogenic composition allows the recipient subject to acquire immune protection, including memory immune protection, against disease, pathology, and/or the symptomes thereof, caused by one or more of the Histoplasma capsulatum, Coccidioides immitis, Mucor circinelloides, Rhizopus oryzae (also called Rhizopus arrhizus), Rhizopus delemar, or Candida auris fungal pathogens.

Methods for administering both single and combination therapies (e.g., concurrently or otherwise) are known to those skilled in the art and are described, for example, in Remington's Pharmaceutical Sciences, 12^{th} edition, Edited by E. W. Martin, Mack Publishing Co. In an embodiment, the antiserum provides a therapeutic, antibody-containing composition that treats disease caused by one or more fungal pathogens as described herein. In another embodiment, the antiserum provides a prophylactic, antibody-containing composition that protects against disease caused by one or more fungal pathogens as described herein. In an embodiment, the isolated antiserum is in a pharmaceutically acceptable composition.

### Additional Methods

At present, there is a dearth of methods as well as reagents to determine if a patient who is asymptomatic for infection by one or more of, or each of, the Histoplasma capsulatum, Coccidioides immitis, Mucor circinelloides, Rhizopus oryzae (also called Rhizopus arrhizus), Rhizopus delemar, or Candida auris fungal pathogens is susceptible, vulnerable, or at risk for infection by one or more of these fungal pathogens. It is currently difficult to plate out the fungal organisms as they may be present in very low amounts, or they may not grow under the culture conditions available for assessing their presence in a subject. Consequently, it is difficult for a medical practitioner and the patient to know whether the patient is actually infected with one or more of Candida auris, Histoplasma capsulatum, Coccidioides immitis, Mucor circinelloides, Rhizopus oryzae (also called Rhizopus arrhizus), or Rhizopus delemar fungal organisms, or if they are likely to become infected, for example, after a medical procedure, surgery, or transplant.

It is also difficult to identify, qualify, or stratify patients who are or who may be susceptible to infection by one or more fungal pathogen and to monitor patients for changes in susceptibility over time, e.g., during recovery from surgery or during immunosuppressive therapies, for example, following organ transplantation, or during chemotherapy treatments, or for reduction or elimination of infection in a patient undergoing treatment for a fungal infection or associated disease over time. The methods described herein provide a viable solution for such medical needs. In addition, methods are provided that allow a patient to be treated with the appropriate or a more directed fungal therapy by stratifying patients based on whether they possess or do not possess antibodies specific for a particular fungal pathogen, thereby deterring disease and/or infection, or based on whether they have or do not have anti-fungal kex peptide antibodies that are specific for the Histoplasma capsulatum, Coccidioides immitis, Mucor circinelloides, Rhizopus oryzae (also called Rhizopus arrhizus), Rhizopus delemar, or Candida auris fungal organisms.

In an embodiment, a method is provided for detecting antibodies against a non-naturally occurring, kex peptide of Histoplasma capsulatum, Coccidioides immitis, Mucor circinelloides, Rhizopus oryzae (also called Rhizopus arrhizus), Rhizopus delemar, or Candida auris in a sample obtained from a subject, in which the method comprises: (a) contacting a biological sample obtained from the subject with a non-naturally occurring, recombinat, kex peptide antigen as set forth in one or more of SEQ ID NOs: 1-6; and (b) detecting the specific binding of the non-naturally occurring, recombinant kex peptide to antibodies in the sample, wherein the detection of binding is indicative of the presence of antibodies against the kex peptide of one or more of the Histoplasma capsulatum, Coccidioides immitis, Mucor circinelloides, Rhizopus oryzae (also called Rhizopus arrhizus), Rhizopus delemar, or Candida auris fungal organisms in the subject's sample. In an embodiment, the kex peptide is attached to a solid support or substrate. In an embodiment, specific binding is detected by performing an immunoassay, e.g., an enzyme linked immunosorbent assay, or other type of immunoassay as known and practiced in the art.

In another embodiment, a method of monitoring or detecting antibodies to a kex peptide antigen of one or more of the Histoplasma capsulatum, Coccidioides immitis, Mucor circinelloides, Rhizopus oryzae (also called Rhizopus arrhizus), Rhizopus delemar, or Candida auris fungal organisms that cause disesae or pathology in a subject who has undergone a transplant or who is to undergo a transplant procedure to determine, for example, whether the subject is protected or will be protected from infection by one or more of the Histoplasma capsulatum, Coccidioides immitis, Mucor circinelloides, Rhizopus oryzae (also called Rhizopus arrhizus), Rhizopus delemar, or Candida auris fungal pathogens, The method involves (a) measuring in a sample obtained from the subject prior to undergoing transplant surgery at a first time point the level of antibodies that bind to a non-naturally occurring, recombinant kex peptide antigen of one or more of Histoplasma capsulatum, Coccidioides immitis, Mucor circinelloides, Rhizopus oryzae (also called Rhizopus arrhizus), Rhizopus delemar, or Candida auris (b) measuring the levels of antibodies that bind to the kex antigen in a sample obtained from the subject at one or more time points after the subject has undergone transplant surgery; and (c) detecting that the subject's sample contains a level of antibodies that specifically binds to the non-naturally occurring, recombinant kex peptide antigen relative to a predetermined or threshold level, or to a control level, wherein a high level of antibodies that bind to the kex peptide antigen in the subject's sample indicates that the subject has produced an immune response against the fungal organism. In an embodiment, antibodies detected in the subject's sample that bind to the kex peptide antigen may serve to protect the subject from disease or pathology caused by or related to infection by Histoplasma capsulatum, Coccidioides immitis, Mucor circinelloides, Rhizopus oryzae (also called Rhizopus arrhizus), Rhizopus delemar, or Candida auris according to the present described methods and compositions.

Repeating the practice of the above-described method over time (at different time intervals or different time periods) allows monitoring of the subject's antibody levels against the kex peptide antigen(s) (i.e., the levels of antibodies that bind the kex peptide antigen) of the different fungal organisms and can inform the medical practitioner or clinician as to whether continued, new, or different treatment of the subject with an appropriate anti-fungal drug or therapy is needed or warranted, or whether no, or a decreased amount of, anti-fungal treatment is warranted, based on the measured titers of antibodies in the subject's sample.

Other embodiments also provide methods for detecting in a subject's biological sample, e.g., blood, serum, plasma, lymph, bronchoalveolar lavage fluid, the presence of antibodies that bind a non-naturally occurring, recombinant kex peptide antigen of Histoplasma capsulatum, Coccidioides immitis, Mucor circinelloides, Rhizopus oryzae (also called Rhizopus arrhizus), Rhizopus delemar, or Candida auris, wherein the levels of antibodies against the kex peptide antigen in the biological sample are determined simultaneously. For example, in one embodiment, the method comprises: (a) contacting a biological sample obtained from the subject with the kex peptide antigens for a period of time sufficient for antibodies specific for the kex peptide antigens to form bound kex peptide antigen-antibody complexes; (b) detecting binding of the kex peptide antigens to the antibodies in the subject's sample, thereby determining the antibody levels in the sample; and (c) comparing the levels of the antibodies in the sample with predetermined threshold values, wherein levels of antibodies that bind to at least one of the kex peptide antigens that are above or below the predetermined threshold values indicate, for example, that the subject has, or does not have, an antibody titer and has, or has not, generated an immune response against the kex peptide antigen derived from one or more of the Histoplasma capsulatum, Coccidioides immitis, Mucor circinelloides, Rhizopus oryzae (also called Rhizopus arrhizus), Rhizopus delemar, or Candida auris fungal organisms. Accordingly, the subject having a measured antibody response (and/or high levels of antibodies that bind specifically to the kex peptide antigen) is protected from disease and pathology associated with infection by one or more of the Histoplasma capsulatum, Coccidioides immitis, Mucor circinelloides, Rhizopus oryzae (also called Rhizopus arrhizus), Rhizopus delemar, or Candida auris fungal organisms.

In another embodiment, methods for assessing antibodies that bind to a kex peptide antigen of one or more of the fungal organisms by Histoplasma capsulatum, Coccidioides immitis, Mucor circinelloides, Rhizopus oryzae (also called Rhizopus arrhizus), Rhizopus delemar, or Candida auris in a subject are provided, in which the methods include: (a) contacting a biological sample obtained from the subject with a composition comprising one or more (a plurality of) kex peptide antigens, e.g., SEQ ID NOs: 1-6 of Histoplasma capsulatum, Coccidioides immitis, Mucor circinelloides, Rhizopus oryzae (also called Rhizopus arrhizus), Rhizopus delemar, or Candida auris for a period of time sufficient to form antibody-kex peptide complexes; (b) detecting binding of the plurality of the kex peptide antigens to antibodies in the sample, thereby detecting the level or titer of anti-kex peptide antibodies in the sample; and (c) comparing the level or titer of the anti-kex peptide antigen antibodies in the biological sample with predetermined threshold values or control values, wherein levels of at least one of the anti-kex peptide antigen antibodies above or below the predetermined threshold values indicates that the subject has, or does not have, respectively, an adequate immune response (antibody response) to protect against or prevent infection by the one or more fungal organisms.

Also provided is a composition comprising a solid substrate and a plurality of recombinant kex peptide antigens of one or more of Histoplasma capsulatum, Coccidioides immitis, Mucor circinelloides, Rhizopus oryzae (also called Rhizopus arrhizus), Rhizopus delemar, or Candida auris immobilized on the substrate. In an embodiment, a kex peptide from each of Histoplasma capsulatum, Coccidioides immitis, Mucor circinelloides, Rhizopus oryzae (also called Rhizopus arrhizus), Rhizopus delemar, or Candida auris is immobilized at a different, indexable, location on the substrate. In other embodiments, a mixture of recombinant kex peptide antigens from two or more, three or more, or four or more, five or more, or all six of Histoplasma capsulatum, Coccidioides immitis, Mucor circinelloides, Rhizopus oryzae (also called Rhizopus arrhizus), Rhizopus delemar, or Candida auris is immobilized at a different, indexible, location on the substrate. The binding of anti-kex peptide antigen antibodies from a sample obtained from a subject previously vaccinated with a non-naturally occurring, recombinant, immunogenic kex peptide antigen can be measured or detected by measuring or detecting complexes of the anti-kex peptide antigen antibodies bound to the recombinant kex peptide antigens localized on the substrate. In an embodiment, the composition is contained in a kit for performing an assay, e.g., an immunoassay, to detect and/or measure the antibody-kex peptide antigen complexes, as well as determine or measure the levels or amounts of antibody(ies) present in the subject's sample.

### Antibodies

As described herein, antisera comprising antibodies that specifically bind to a non-naturally occurring, recombinant kex (kex1) peptide antigen and that cross-react with a kex peptide antigen of one or more of Histoplasma capsulatum, Coccidioides immitis, Mucor circinelloides, Rhizopus oryzae (also called Rhizopus arrhizus), Rhizopus delemar, or Candida auris fungal organisms, to provide immune protection against disease, pathology, and/or the symptoms thereof, caused by one, or more than one of these fungal pathogens are useful in therapeutic methods. For example, isolated antiserum containing antibodies that target and/or inhibit or neutralize the activity of the kex peptide and that cross-react with two or more of the Histoplasma capsulatum, Coccidioides immitis, Mucor circinelloides, Rhizopus oryzae (also called Rhizopus arrhizus), Rhizopus delemar, or Candida auris fungal pathogens is particularly useful in the methods as described herein. In particular embodiments, methods of using isolated antiserum (or immune plasma) comprising antibodies, or antigen binding fragments thereof, that specifically bind the the kex peptide antigens and that cross-react with Histoplasma capsulatum, Coccidioides immitis, Mucor circinelloides, Rhizopus oryzae (also called Rhizopus arrhizus), Rhizopus delemar, or Candida auris kex peptide antigens can be provided for the treatment, protection against, or prevention of disease and pathology associated with infection by these fungal pathogens, such as pulmonary diseases and disorders of various types, pneumonia, COPD, and the like.

Methods of preparing antibodies are well known to those of ordinary skill in the science of immunology. As used herein, the term “antibody” refers not only to intact antibody molecules, but also to fragments or portions of antibody molecules that retain immunogen-binding ability. Such fragments and portions are also well known in the art and are regularly employed both in vitro and in vivo. Accordingly, as used herein, the term “antibody” refers not only to intact immunoglobulin molecules, but also to the well-known active fragments F(ab′)₂, and Fab. F(ab′)₂, and Fab fragments that lack the Fc fragment of an intact antibody, clear more rapidly from the circulation, and may have less nonspecific tissue binding than an intact antibody (Wahl et al., J. Nucl. Med. 24:316-325 (1983)). The antibodies may comprise whole native antibodies, bispecific antibodies; chimeric antibodies; Fab, Fab′, single chain V region fragments (scFv), fusion polypeptides, and unconventional antibodies.

Unconventional antibodies include, but are not limited to, nanobodies, linear antibodies (Zapata et al., Protein Eng. 8(10): 1057-1062, (1995)), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody, because small molecules of ~60-100 kDa in size provide faster blood clearance and rapid tissue uptake. See, e.g., Power et al., (Generation of recombinant multimeric antibody fragments for tumor diagnosis and therapy, Methods Mol Biol, 207, 335-50, (2003); and Wu et al., Anti-carcinoembryonic antigen (CEA) diabody for rapid tumor targeting and imaging, Tumor Targeting, 4, 47-58, (1999)).

Various techniques for making and using unconventional antibodies have been described. Bispecific antibodies produced using leucine zippers are described by Kostelny et al. (J. Immunol. 148(5):1547-1553, (1992)). Diabody technology is described by Hollinger et al. (Proc. Natl. Acad. Sci. USA 90:6444-6448, (1993)). Another strategy for making bispecific antibody fragments using single-chain Fv (sFv) diners is described by Gruber et al. (J. Immunol. 152:5368, (1994)). Trispecific antibodies are described by Tutt et al. (J. Immunol. 147:60, (1991)). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, (1988)). See, also, U.S. Pat. Nos. 5,091,513, 5,132,405 and 4,956,778; and U.S. Patent Publication Nos. 20050196754 and 20050196754.

In various embodiments, an antiserum (isolated antiserum) contains monoclonal or polyclonal antibodies, or antigen binding fragments thereof, that specifically bind to the non-naturally occurring, recombinant, kex peptide antigens of Histoplasma capsulatum, Coccidioides immitis, Mucor circinelloides, Rhizopus oryzae (also called Rhizopus arrhizus), Rhizopus delemar, or Candida auris. Also encompassed are methods of obtaining or isolating the antibodies from immune serum (antiserum) or immune plasma and producing hybrid or chimeric antibodies therefrom. In such hybrid or chimeric antibodies, one pair of heavy (H) and light (L) chains is obtained from a first antibody, while the other pair of heavy and light chains is obtained from a different second antibody. Such hybrids or chimeric antibodies may also be formed using humanized heavy chains (HC) and light chains (LC). Methods for isolating antibodies and producing hybrid or chimeric antibodies are known and practiced by those having skill in the art.

In general, intact antibodies are said to contain “Fc” and “Fab” regions. The Fc regions are involved in complement activation and are not involved in antigen binding. An antibody from which the Fc region has been enzymatically cleaved, or which has been produced without the Fc region, designated an “F(ab′)₂” fragment, retains both antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies (and immune serum or plasma containing antibodies) can be produced or generated by any of the methods known in the art utilizing soluble polypeptides, or immunogenic fragments thereof, (e.g., a recombinant immunogenic kex peptide antigen) as an immunogen. One method of obtaining antibodies is to immunize suitable host animals or subjects with the immunogen, or a polynucleotide encoding the immunogen, and to follow standard procedures for polyclonal or monoclonal antibody production. In brief, the immunization will facilitate presentation of the immunogen (or immunogenic fragments of the immunogen) on the cell surface. Immunization of a suitable host can be carried out in a number of ways. By way of example, nucleic acid sequences encoding immunogenic kex peptide antigens can be provided to the host in a delivery vehicle (or a molecular expression construct) that is taken up by immune cells of the host. The cells will, in turn, process and appropriately express the kex peptide antigen in a manner that generates an immunogenic response in the host. In embodiments, the kex peptide antigen may be expressed by the delivery vehicle or expression construct. In other exemplary embodiments, nucleic acid sequences encoding the kex peptide antigen may be expressed in cells in vitro, and the expressed, recombinant kex peptide antigen products may be isolated and used as immunogens to raise anti-kex peptide antibodies and to generate an anti-kex antiserum in a suitable immunized host.

Alternatively, antibodies against recombinant, immunogenic kex peptide antigen may, if desired, be derived from an antibody phage display library. A bacteriophage is capable of infecting and reproducing within bacteria, which can be engineered, when combined with human immunoglobulin (antibody) genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface.

Antibodies made by any method known in the art can then be purified from an immunized host. Antibody purification methods include, without limitation, salt precipitation (for example, with ammonium sulfate), ion exchange chromatography (for example, on a cationic or anionic exchange column, preferably run at neutral pH and eluted with step gradients of increasing ionic strength), gel filtration chromatography (including gel filtration HPLC) and chromatography on affinity resins such as protein A, protein G, hydroxyapatite, or anti-immunoglobulin.

In certain aspects, antibodies can be conveniently produced from hybridoma cells engineered to express the antibody. Methods of making hybridomas are well known in the art. The hybridoma cells can be cultured in a suitable medium into which antibodies produced by the hybridoma cells are secreted; the spent medium can be used as an antibody source. Polynucleotides encoding the antibody of interest can, in turn, be obtained from the hybridoma cells that produce the antibody, and then the antibody may be produced synthetically or recombinantly from these nucleic acid sequences. To produce large amounts of antibody, it is generally more convenient to obtain an ascites fluid. The method of raising ascites fluid generally comprises injecting hybridoma cells into an immunologically naive histocompatible or immunotolerant mammal, especially a mouse. The mammal may be primed for ascites production by prior administration of a suitable composition (e.g., Pristane). Ascites fluid containing antibodies, typically in high concentration, can be obtained from the peritoneal fluid of the animal that harbors the injected hybridoma cells.

Monoclonal antibodies (Mabs can also be “humanized” by methods known in the art. “Humanized” antibodies are antibodies in which at least part of the sequence has been altered from its initial form to render it more like immunoglobulins derived from a human source. Techniques to humanize antibodies are particularly useful when antibodies are generated in a non-human animal (e.g., mice, rats). Nonlimiting examples of methods for humanizing a murine antibody are provided in U.S. Pat. Nos. 4,816,567, 5,530,101, 5,225,539, 5,585,089, 5,693,762 and 5,859,205, the entire contents of which are incorporated by reference herein.

In an embodiment of the foregoing, one or more antibodies or antigen binding fragments thereof generated against non-naturally occurring, recombinant kex peptide antigens of Histoplasma capsulatum, Coccidioides immitis, Mucor circinelloides, Rhizopus oryzae (also called Rhizopus arrhizus), Rhizopus delemar, or Candida auris to provide immune protection against disease or pathology caused by or associated with one or more of these fungal pathogens in a subject in need thereof. Such antibodies may be isolated or purified from an antiserum as described herein, or they may be generated, e.g., by recombinant molecular biology techniques, purified and formulated for pharmaceutical use in a subject in need. Such a formulation of antibodies may have immune protective properties similar to those afforded by an isolated antiserum comprising anti-fungal kex peptide antigen antibodies as described herein.

### Vaccines

A vaccine is a biological preparation that provides active, acquired immunity (e.g., protective immunity) in a subject to a particular disease. A vaccine typically contains an agent that resembles a disease-causing pathogenic agent, e.g., a microorganism, a fungus, etc., and is often made from a weakened or killed form of the agent, or a toxin, or a surface protein or peptide antigen of the agent. After administration of the vaccine to a subject, the agent is expressed and recognized as foreign (or “non-self”) to the subject and stimulates the subject's immune system to mount an immune response (a B cell (antibody) and/or a T cell (cellular) immune response) to destroy the agent. In addition, cells (e.g., B cells) of the immune system that are exposed to the vaccinating agent retain a memory of the agent, such that the agent is recognized and destroyed by the memory cells upon a later or subsequent encounter. Vaccines can be prophylactic (e.g., to protect against, prevent, or ameliorate the effects of a future infection by a pathogen or fungal pathogen), or therapeutic (e.g., to treat disease or infections caused by or associated with pathogens, e.g., fungal pathogens, or disease-causing agents upon or after a subject has been infected with or has encountered or been exposed to a pathogen).

While many vaccines are prepared from an attenuated version of a pathogen or from inactivated disease-causing organisms, or a suitable part of such pathogens or organisms, such as a toxin, protein/peptide antigen, or deleterious enzyme, the antigen to which the immune system responds frequently constitutes a relatively small number of amino acids, such as a peptide (e.g., a non-naturally occurring, recombinant form of a fungal kex peptide antigen). A protein or peptide part of a pathogen may constitute a vaccine. A peptide vaccine is any peptide which serves to immunize an organism (elicit an immune response or a protective immune response, such as an antibody (B cell) response and/or an immune cell (T cell) response in the immunized organism) against a pathogen. In embodiments, the peptide antigen may be a non-naturally occurring, recombinant kex peptide antigen of Histoplasma capsulatum, Coccidioides immitis, Mucor circinelloides, Rhizopus oryzae (also called Rhizopus arrhizus), Rhizopus delemar, or Candida auris. In an embodiment, a vaccine comprising a non-naturally occurring, recombinant kex peptide antigen may be used to provide immune protection against the fungal organism that expresses the kex peptide antigen, or against other fungal organisms having kex peptide antigens that elicit production of cross-reactive antibodies following administration to a recipient subject in need.

For non-attenuated vaccines, the peptide sequences that trigger a protective immune response are identified, and synthetic (or recombinantly-produced) versions of the peptides are employed as the vaccine substance. Because they are non-naturally occurring and synthetic, peptide vaccines pose little to no risk of mutation or reversion, and little or no risk of contamination by pathogenic or toxic substances. Moreover, chemical manipulation or modification of the peptide structure may result in increased stability and decreased unwanted side effects or adverse effects that may be associated with a native protein or peptide sequence.

Synthetically or recombinantly produced peptide antigens can be readily prepared in large amounts as components of vaccines. Such substances may also expose parts of a protein antigen that are not recognized by the immune system during a natural infection, possibly as a result of masking or post-translational modifications of proteins. Sequencing new strains and serotypes of microorganisms, fungal pathogens and other pathogenic organisms allows for rapid modification of peptide antigens to generate strain-specific immune responses, particularly against an antigenic epitope that is recognized and targeted by antibodies and cells of the host's or recipient's immune system. In some cases, modelling of three-dimensional epitopic or antigenic sites of a pathogen may be employed to generate synthetically the correct epitopic or antigenic site(s) on peptide antigens.

In an aspect, a vaccine (or an immunogenic composition) is provided, which comprises a synthetically (recombinantly) produced peptide, i.e., a kex (kex1) peptide antigen of Histoplasma capsulatum, Coccidioides immitis, Mucor circinelloides, Rhizopus oryzae (also called Rhizopus arrhizus), Rhizopus delemar, or Candida auris, as set forth in SEQ ID NOs: 1-6, respectively, that is nonidentical, but immunologically targetable, among several different types of fungal pathogens (e.g., the Histoplasma capsulatum, Coccidioides immitis, Mucor circinelloides, Rhizopus oryzae (also called Rhizopus arrhizus), Rhizopus delemar, or Candida auris fungal pathogens) and is useful for treating or potecting against disease or pathology and/or the symptoms thereof caused by or associated with one or more of these fungal pathogens after administration (immunization) to a subject. In an embodiment, a kex peptide vaccine or immunogenic composition from a non-naturally occurring, recombinant kex peptide antigen, when used to immunize an individual, elicits an immune response in the form of the production of antiserum (or immune plasma) containing cross-reactive antibodies which protect against some or all of the etiologically distinct fungal pathogens Histoplasma capsulatum, Coccidioides immitis, Mucor circinelloides, Rhizopus oryzae (also called Rhizopus arrhizus), Rhizopus delemar, or Candida auris. Accordingly, an antiserum or immune plasma generated by a vaccine or immunogenic composition comprising a non-naturally occurring, recombinant fungal kex peptide antigen may be used as a sole therapeutic or protective agent needed to treat or protect against disease or pathology and/or the symptoms thereof caused by or associated with infection by one or more than one different fungal pathogen, namely, the Histoplasma capsulatum, Coccidioides immitis, Mucor circinelloides, Rhizopus oryzae (also called Rhizopus arrhizus), Rhizopus delemar, or Candida auris fungal pathogens. In some embodiments, the subject is further treated or protected against disease or pathology and/or the symptoms thereof caused by or associated with infection by Pneumocystis hominis, Aspergillus fumigatus, Candida albicans and/or Cryptococcus neoformans. In an embodiment, the antiserum generated by the described kex peptide antigen vaccine is isolated. In an embodiment, the isolated antiserum is used in a pharmaceutical composition.

In some aspects, a genetic vaccine is provided. A genetic vaccine is any vaccine that comprises a polynucleotide sequence encoding an immunogen, wherein the immunogen, once expressed, serves to immunize an organism. Administration of a genetic vaccine elicits an immune response or a protective immune response, such as an antibody (B cell) response and/or an immune cell (T cell) response in the immunized organism (such as a human patient or a mammalian recipient) against a pathogen after the immunogen is expressed in a cell. In some embodiments, the genetic vaccine provides a polynucleotide sequence encoding an immunogenic kex peptide antigen, such as a kex peptide antigen of Histoplasma capsulatum, Coccidioides immitis, Mucor circinelloides, Rhizopus oryzae (also called Rhizopus arrhizus), Rhizopus delemar, or Candida auris. In some embodiments, the peptide is a non-naturally occurring, recombinant peptide. In some embodiments, the genetic vaccine provides a polynucleotide sequence encoding more than one immunogenic kex peptide antigen. In some embodiments, the polynucleotide encoding the immunogenic kex peptide antigen is harbored in a vector having elements, such as promoters and enhancers, to facilitate expression of the encoded kex peptide antigen. The nucleic acid in a genetic vaccine may be integrated into the subject's genome, wherein expression of the immunogen may be driven by an endogenous promoter or a promoter encoded by the inserted nucleic acid. The polynucleotide encoding the immunogen in a genetic vaccine may be a DNA polynucleotide or a RNA polynucleotide. The polynucleotide may include a nucleotide analog, which can inhibit degradation of the polynucleotide.

### Pharmaceutical Compositions

Also featured herein are methods for treating or protecting aginst disease, pathology, and/or the symptoms thereof associated with or caused by infection of a subject by one or more fungal pathogens, namely, Histoplasma capsulatum, Coccidioides immitis, Mucor circinelloides, Rhizopus oryzae (also called Rhizopus arrhizus), Rhizopus delemar, or Candida auris fungal pathogens. The methods include administering to a subject in need thereof an immunologically effective amount of an immunogenic composition comprising a non-naturally occurring, recombinant, immunogenic kex peptide antigen, a polynucleotide encoding the kex peptide antigen, or an isolated antiserum generated in response to immunization of a recipient individual with the non-naturally occurring, recombinant, immunogenic kex peptide antigen, wherein the antiserum contains antibodies which cross-react with, cross-treat, and/or protect the subject from disease associated with infection by one or more of the fungal pathogens Histoplasma capsulatum, Coccidioides immitis, Mucor circinelloides, Rhizopus oryzae (also called Rhizopus arrhizus), Rhizopus delemar, or Candida auris. In an embodiment, the isolated antiserum is used in a pharmaceutical composition.

Typically, the carrier or excipient for an immunogenic composition or vaccine as described herein is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is affected according to protocols established in the art. Generally, a carrier or excipient is selected to minimize allergic and other undesirable effects, and to suit the particular route of administration, e.g., subcutaneous, intramuscular, intranasal, and the like. Such methods also include administering an adjuvant, such as an oil-in-water emulsion, a saponin, a cholesterol, a phospholipid, a CpG, a polysaccharide, variants thereof, and a combination thereof, with a composition as described herein. Optionally, a formulation for prophylactic administration also contains one or more adjuvants for enhancing the immune response to an antigen or immunogen, such as a pan-fungal kex peptide antigen or immunogen. Suitable adjuvants include, without limitation, complete Freund's adjuvant, incomplete Freund's adjuvant, saponin, alpha-galactosylceramide (α-GC), mineral gels such as aluminum hydroxide, surface active substances such as lysolecithin, pluronic polyols, polyanions, peptides, oil or hydrocarbon emulsions, bacille Calmette-Guerin (BCG), Corynebacterium parvum, and the synthetic adjuvants QS-21 and MF59. In an embodiment, the isolated antiserum is used in a pharmaceutical composition.

The administration of an immunogenic composition comprising a non-naturally occurring, recombinant, immunogenic kex peptide antigen of Histoplasma capsulatum, Coccidioides immitis, Mucor circinelloides, Rhizopus oryzae (also called Rhizopus arrhizus), Rhizopus delemar, or Candida auris (e.g., a peptide sequence as set forth in any one of SEQ ID NOs: 1-6), a polynucleotide encoding the kex peptide antigen, or an antiserum, such as an isolated antiserum, monoclonal or polyclonal antibodies, generated against the kex peptide antigen as a therapeutic for the treatment or protection against disease or pathology associated with the Histoplasma capsulatum, Coccidioides immitis, Mucor circinelloides, Rhizopus oryzae (also called Rhizopus arrhizus), Rhizopus delemar, or Candida auris fungal pathogen, may be by any suitable means that results in a concentration of the therapeutic that, combined with other components, if desired, is effective in ameliorating, reducing, eliminating, abating, stabilizing, or eliminating disease or disease symptoms in a subject. The therapeutic may be administered systemically, for example, formulated in a pharmaceutically acceptable composition or buffer such as physiological saline. Preferable routes of administration include, for example, subcutaneous, intravenous, intraperitoneally, intramuscular, intrathecal, or intradermal injections that provide continuous, sustained levels of the therapeutic in the subject. The amount of the therapeutic to be administered varies depending upon the manner of administration, the age and body weight of the subject, and with the clinical symptoms of the fungal infection or associated disease. Generally, amounts will be in the range of those used for other agents used in the treatment of pulmonary disease or dysfunction, although in certain instances lower amounts may be suitable because of the increased range of protection and treatment afforded by the therapeutic. A composition is administered at a dosage that ameliorates, decreases, diminishes, abates, alleviates, or eliminates the effects of infection by the fungal pathogen or disease and/or the symptoms thereof), as determined by a method known to one skilled in the art. In an embodiment, an isolated antiserum is administered or provided to a recipient subject at or near a site of the infection or colonization by the pathogenic fungal organism or organisms.

In embodiments, a therapeutic or prophylactic treatment agent may be contained in any appropriate amount in any suitable carrier substance and is generally present in an amount of 1-95% by weight of the total weight of the composition. The composition may be provided in a dosage form that is suitable for parenteral (e.g., subcutaneous, intravenous, intramuscular, intrathecal, or intraperitoneal) administration route. The pharmaceutical compositions may be formulated according to conventional pharmaceutical practice (see, e.g., Remington: The Science and Practice of Pharmacy (20^{th} ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions may in some cases be formulated to release the active agent substantially immediately upon administration or at any predetermined time or time period after administration. The latter types of compositions are generally known as controlled release formulations, which include (i) formulations that create a substantially constant concentration of a therapeutic agent or drug within the body over an extended period of time; (ii) formulations that after a predetermined lag time create a substantially constant concentration of a therapeutic agent or drug within the body over an extended period of time; (iii) formulations that sustain action during a predetermined time period by maintaining a relatively constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with an organ, such as the heart, kidney, or colon; (v) formulations that allow for convenient dosing, such that doses are administered, for example, once every one or two weeks; and (vi) formulations that target a disease using carriers or chemical derivatives to deliver the therapeutic agent or drug to a particular cell type. For some applications, controlled release formulations obviate the need for frequent dosing during the day to sustain a therapeutic level in plasma, serum, or blood. In an embodiment, an isolated antiserum may be formulated with one or more additional components for administration to a subject.

Any of a number of strategies can be pursued to obtain controlled release, in which the rate of release outweighs the rate of metabolism of the therapeutic agent or drug in question. In one example, controlled release is obtained by appropriate selection of various formulation parameters and ingredients, including, e.g., various types of controlled release compositions and coatings. Thus, the therapeutic agent or drug may be formulated with appropriate excipients into a pharmaceutical composition that, upon administration, releases the therapeutic agent or drug in a controlled manner. Examples include single or multiple unit tablet or capsule compositions, oil solutions, suspensions, emulsions, microcapsules, microspheres, molecular complexes, nanoparticles, patches, and liposomes.

A pharmaceutical composition may be administered parenterally by injection, infusion, or implantation (subcutaneous, intravenous, intramuscular, intraperitoneal, intrathecal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, noted supra.

Compositions for parenteral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a disease or dysfunction, such as pulmonary disease or dysfunction, the composition may include suitable parenterally acceptable carriers and/or excipients. In some cases, an active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, a pharmaceutical composition comprising an active therapeutic (e.g., an immunogenic composition comprising a non-naturally occurring, recombinant, immunogenic kex peptide antigen of Histoplasma capsulatum, Coccidioides immitis, Mucor circinelloides, Rhizopus oryzae (also called Rhizopus arrhizus), Rhizopus delemar, or Candida auris, a polynucleotide encoding the kex peptide antigen, or an isolated anti-fungal antiserum generated in response to the kex peptide antigen as described herein is formulated for intravenous delivery, e.g., intravenous (IV) injection or intrathecal delivery. To prepare such a composition, the suitable therapeutic(s) are dissolved or suspended in a parenterally acceptable liquid vehicle, excipient, or solvent. Among acceptable vehicles and solvents that may be employed are, for example, water; water adjusted to a suitable pH by the addition of an appropriate amount of hydrochloric acid, sodium hydroxide or a suitable buffer; 1,3-butanediol; Ringer's solution; and isotonic sodium chloride solution and dextrose solution. The aqueous formulation may also contain one or more preservatives (e.g., methyl, ethyl or n-propyl p-hydroxybenzoate). In cases in which one of the agents is only sparingly or slightly soluble in water, a dissolution enhancing or solubilizing agent can be added, or the solvent may include 10-60% w/w of propylene glycol or the like. In some cases, the antiserum is an isolated antiserum.

Kits and Compositions for Detecting and/or Quantifying Antibodies that React with Fungal-Derived, Immunogenic Kex Peptide Antigens

Kits and compositions are provided that advantageously allow for the detection and/or quantification of the presence of antibodies directed against the kex peptide antigen of one or more of Histoplasma capsulatum, Coccidioides immitis, Mucor circinelloides, Rhizopus oryzae (also called Rhizopus arrhizus), Rhizopus delemar, or Candida auris fungal pathogens, or the levels of such one or more antibodies that may be present, in a subject's sample (e.g., blood or serum). In an embodiment, the subject is a human patient. In an embodiment, the patient has undergone a transplant, e.g., an organ or tissue transplant, or is to undergo a transplant, and thus may be at higher risk for infection by one or more fungal pathogens. In an embodiment, the transplant patient, or the patient to undergo a transplant, is immunosuppressed and/or is otherwise treated with drugs to reduce the likelihood of rejection of the transplanted organ or tissue, thereby making the patient more vulnerable or susceptible to infection and/or disease caused by one or more of the Histoplasma capsulatum, Coccidioides immitis, Mucor circinelloides, Rhizopus oryzae (also called Rhizopus arrhizus), Rhizopus delemar, or Candida auris fungal pathogens. In an embodiment, the patient has received, or is to receive, a transplant of an organ selected from kidney, liver, heart, bone marrow, pancreas, lung, etc.

Such kits as described herein fulfill a long-felt need in the art for detecting or qualifying whether any patient, but particularly a transplant patient, has adequate levels (titer) of anti-fungal pathogen antibodies to ensure that the patient does not become infected with one or more fungal pathogens as described herein, for example, during a hospital stay, or during or following a medical procedure or treatment (e.g., surgery or transplant), performed either on in-patient or an out-patient basis. At present, because of a lack of appropriate reagents and assays, it is difficult to assess whether a patient who is to undergo a medical procedure or surgery, in particular, an immunosuppressed patient who is to undergo a transplant procedure, or a patient who is to initiate other immunosuppressive therapies, will contract a fungal infection, e.g., infection and/or disease caused by one or more of the Histoplasma capsulatum, Coccidioides immitis, Mucor circinelloides, Rhizopus oryzae (also called Rhizopus arrhizus), Rhizopus delemar, or Candida auris fungal pathogens following or during immunosuppressive therapies and treatments. The use of a kit with which a patient's sample can be tested to determine if the patient has an antibody titer against one or more of these fungal pathogens (e.g., a high or a low antibody titer against one or more of the foregoing fungal pathogens) would greatly enhance the success of the patient's post-surgical or post-transplant recovery and directed treatment. For example, if, following testing of a patient's sample (e.g., a blood or serum sample from a transplant patient) using a kit as described herein, the patient is determined to have a low, negligible, or no antibody (antiserum) titer against one or more of the fungal pathogens, in particular, against the kex peptide antigen of one or more of the Histoplasma capsulatum, Coccidioides immitis, Mucor circinelloides, Rhizopus oryzae (also called Rhizopus arrhizus), Rhizopus delemar, or Candida auris fungal pathogens, it could be surmised that the patient would not be naturally protected against a possible or real infection by one or more of the Histoplasma capsulatum, Coccidioides immitis, Mucor circinelloides, Rhizopus oryzae (also called Rhizopus arrhizus), Rhizopus delemar, or Candida auris fungal pathogens.

A kit as described herein would allow the tester and the patient to determine and know if the patient's sample (serum sample) contains antibodies against one or more of the kex peptide antigens of the Histoplasma capsulatum, Coccidioides immitis, Mucor circinelloides, Rhizopus oryzae (also called Rhizopus arrhizus), Rhizopus delemar, or Candida auris fungal organisms. Should the results obtained from the use of the kit indicate that the patient has no specific anti-fungal kex peptide antigen antibodies, or a low titer of such antibodies (e.g., no specific anti-fungal kex peptide antigen antibodies in serum), directed to a specific fungal kex peptide antigen, the patient would be identified as potentially vulnerable or susceptible to infection to a particular fungal pathogen(s) and could then be administered the appropriate anti-fungal treatment for the specific fungal pathogen(s) against which the patient has no, or negligible, specific antibodies, or a reduced antibody titer. In an embodiment, the patient is administered a prophylactic anti-fungal treatment or therapy. In an embodiment, the treatment comprises administering to the patient an appropriate drug or medication that is best designed to treat a disease or pathology, and/or the symptoms thereof associated with infection by a specific fungal pathogen or by one or more fungal pathogens, namely, Histoplasma capsulatum, Coccidioides immitis, Mucor circinelloides, Rhizopus oryzae (also called Rhizopus arrhizus), Rhizopus delemar, or Candida auris. In an embodiment, the treatment comprises administering to the patient a composition as described herein comprising a non-naturally occurring, recombinant, immunogenic kex peptide antigen, or a polynucleotide encoding the kex peptide antigen to generate a cross-reactive (cross-protective) antibody immune response in the patient, thereby reducing or eliminating disease or pathology associated with infection by one or more of the Histoplasma capsulatum, Coccidioides immitis, Mucor circinelloides, Rhizopus oryzae (also called Rhizopus arrhizus), Rhizopus delemar, or Candida auris pathogenic fungal organisms. Antibodies produced against a non-naturally occurring, recombinant kex peptide antigen of one or more of the Histoplasma capsulatum, Coccidioides immitis, Mucor circinelloides, Rhizopus oryzae (also called Rhizopus arrhizus), Rhizopus delemar, or Candida auris fungal organisms may cross-react with a kex peptide antigen of another of the fungal organisms, namely, Histoplasma capsulatum, Coccidioides immitis, Mucor circinelloides, Rhizopus oryzae (also called Rhizopus arrhizus), Rhizopus delemar, or Candida auris, thereby conferring protection (cross-protection) against more than one pathogenic fungal organisms in the patient.

In an embodiment, a kit is provided for detecting, or quanitfing the levels of, antibodies directed against the fungal kex peptide antigen and that cross-react with one or more of the Histoplasma capsulatum, Coccidioides immitis, Mucor circinelloides, Rhizopus oryzae (also called Rhizopus arrhizus), Rhizopus delemar, or Candida auris fungal organisms in a patient sample, in which the kit comprises a substrate having attached thereto a non-naturally occurring, recombinant, kex peptide antigen as set forth in any one or more of SEQ ID NOs: 1-6, derived from one or more of the Histoplasma capsulatum, Coccidioides immitis, Mucor circinelloides, Rhizopus oryzae (also called Rhizopus arrhizus), Rhizopus delemar, or Candida auris fungal organisms for measuring the level(s) of antibodies in the sample which bind or immunologically react with the kex peptide, onto which a biological sample obtained from a patient is applied; and a labeled detection molecule for detecting and measuring the level(s) of antibodies that bind to the kex peptide antigen affixed to the substrate. In an embodiment, detecting anti-fungal kex peptide antibodies in the sample or measuring the level(s) of such antibodies present in the patient's sample is compared to a positive and/or a negative control. In an embodiment, detecting anti-fungal kex peptide antigen antibodies in the sample or measuring the level(s) of such antibodies present in the patient's sample is compared to a cutoff value. In an embodiment, the substrate has attached thereto a non-naturally occurring, recombinant, kex peptide antigen derived from each of Histoplasma capsulatum, Coccidioides immitis, Mucor circinelloides, Rhizopus oryzae (also called Rhizopus arrhizus), Rhizopus delemar, or Candida auris. In an embodiment, the substrate has attached thereto non-naturally occurring, recombinant, kex peptide antigens of two, three, four, or five of Histoplasma capsulatum, Coccidioides immitis, Mucor circinelloides, Rhizopus oryzae (also called Rhizopus arrhizus), Rhizopus delemar, or Candida auris in different combinations. In an embodiment, the detection of antibodies in the sample that bind to the fungal kex peptide antigens is performed using an immunoassay, such as an ELISA, or multiplex assay as described herein. In an embodiment, the ELISA detects a complex between a fungal kex peptide antigen bound to an anti-fungal kex peptide antigen antibody present in the sample. In an embodiment, the detection of antibodies in the sample that bind to the fungal kex peptide antigen is performed using an immunosorbent assay (e.g., ELISA), by immunoprecipitation, by immunoblotting, or a combination thereof.

Also provided are kits comprising reagents that allow for assessing, measuring, evaluating or detecting antibodies directed against a non-naturally occurring, recombinant fungal kex peptide antigen. Such antibodies may be contained in a biological sample obtained from a subject undergoing testing, assessment, or evaluation using the kit. In particular, the biological sample may be a blood, serum, plasma, urine, cerebrospinal fluid, sputum, bronchiolar lavage, tears, saliva, or semen sample, or tissue or cell sample obtained from a subject. In particular, the reagents of the kit comprise non-naturally occurring, recombinant fungal kex peptides of one or more of Histoplasma capsulatum, Coccidioides immitis, Mucor circinelloides, Rhizopus oryzae (also called Rhizopus arrhizus), Rhizopus delemar, or Candida auris fungal pathogens.

In a specific embodiment, the kit is provided as an enzyme linked immunosorbent assay (ELISA) kit comprising a non-naturally occurring, recombinant, fungal kex peptide antigen. In another embodiment, the provided kit allows for the detection of cross-reactive antibodies, wherein the antibodies are produced by immunization with the non-naturally occurring, recombinant pan-fungal kex peptide 2 as set forth in SEQ ID NO: 7, and the antibodies cross-react with a kex peptide antigen of one or more of the Histoplasma capsulatum, Coccidioides immitis, Mucor circinelloides, Rhizopus oryzae (also called Rhizopus arrhizus), Rhizopus delemar, or Candida auris fungal pathogens. In another embodiment, the provided kit allows for the detection of cross-reactive antibodies, wherein the antibodies are produced by immunization with one or more non-naturally occurring, recombinant fungal kex peptide antigens as set forth in SEQ ID NOs: 1-6, and the elicited antibodies react with or cross-react with a kex peptide antigen of one or more of the Histoplasma capsulatum, Coccidioides immitis, Mucor circinelloides, Rhizopus oryzae (also called Rhizopus arrhizus), Rhizopus delemar, or Candida auris fungal pathogens. In such embodiments, the kit is provided as an ELISA kit comprising the kex peptide antigens of Histoplasma capsulatum, Coccidioides immitis, Mucor circinelloides, Rhizopus oryzae (also called Rhizopus arrhizus), Rhizopus delemar, or Candida auris attached to a solid support or substrate. The peptides attached to the substrate thus perform as “capture” reagents that bind to antibodies present in the sample obtained from a subject undergoing testing.

By way of example, the ELISA kit may comprise a solid support, such as a chip, microtiter plate comprising many wells (e.g., a 96-well plate), bead, or resin having the kex peptide antigen capture reagents attached thereon. In one embodiment, the kit comprises a recombinant kex peptide antigen derived from each of Histoplasma capsulatum, Coccidioides immitis, Mucor circinelloides, Rhizopus oryzae (also called Rhizopus arrhizus), Rhizopus delemar, or Candida auris attached independently to discrete areas or components of solid substrates or supports, for example, the recombinant kex peptide antigens of each fungal organism are attached to separate and discrete wells of a microtiter plate or are independently attached to beads to produce populations of beads having the recombinant kex peptide antigens from each of Histoplasma capsulatum, Coccidioides immitis, Mucor circinelloides, Rhizopus oryzae (also called Rhizopus arrhizus), Rhizopus delemar, or Candida auris attached. In another embodiment, the kit comprises a combination or mixture of the recombinant kex peptide antigens derived from Histoplasma capsulatum, Coccidioides immitis, Mucor circinelloides, Rhizopus oryzae (also called Rhizopus arrhizus), Rhizopus delemar, or Candida auris attached to an area or component of the solid substrate or support, for example, the recombinant kex peptide antigens of all of Histoplasma capsulatum, Coccidioides immitis, Mucor circinelloides, Rhizopus oryzae (also called Rhizopus arrhizus), Rhizopus delemar, or Candida auris are attached to a single well of a microtiter plate or to a single bead. In a further embodiment, the kit comprises a combination of one, two or more, three or more, four or more, or five or more of the recombinant kex peptide antigens derived from Histoplasma capsulatum, Coccidioides immitis, Mucor circinelloides, Rhizopus oryzae (also called Rhizopus arrhizus), Rhizopus delemar, or Candida auris attached to a given area of a solid substrate or support, such as a single well of a microtiter plate.

In embodiments, in the ELISA platform, a well of a microtiter plate may have attached thereto a non-naturally occurring, recombinant, fungal kex peptide antigen of any one of, any two of, any three of, any four of, any five of, or all six of the Histoplasma capsulatum, Coccidioides immitis, Mucor circinelloides, Rhizopus oryzae (also called Rhizopus arrhizus), Rhizopus delemar, or Candida auris fungal organisms. The kit may further comprise a means for detecting the peptides or any antibodies bound thereto, e.g., detectable antibodies, a secondary antibody-signal complex, such as horseradish peroxidase (HRP)-conjugated goat anti-rabbit IgG antibody or tetramethyl benzidine (TMB) as a substrate for HRP.

In another embodiment, the kit may be provided as an immunochromatography strip comprising a membrane on which the one, two, three, four, five or six recombinant, kex peptide antigens of Histoplasma capsulatum, Coccidioides immitis, Mucor circinelloides, Rhizopus oryzae (also called Rhizopus arrhizus), Rhizopus delemar, or Candida auris are immobilized, either at discrete loci on the membrane or in combination at one locus or multiple loci of the membrane, and a means for detecting the binding of antibody in a test sample, e.g., detectably labeled kex peptides, or gold particle bound secondary antibodies, in which the membrane may be a nitrocellulose-based (NC) membrane, a PVDF membrane, or other suitable type of membrane used in the art. The kit may comprise a plastic plate or substrate onto which a sample is applied and immobilized detection agents, such as detectably labeled kex peptides, e.g., gold particle-bound peptides temporally spaced and immobilized on the substrate, e.g., a glass fiber filter or a nitrocellulose membrane, or a labeled detection agent that can detect a complex of antibody bound to the recombinant fungal kex peptide antigen in one or more bands on the substrate. In such a platform, a continuous capillary flow of sample, e.g., blood or serum, is maintained over the detection reagents immobilized on the substrate such that sample antibody bound to labeled kex peptide antigen or sample antibody complexed to a kex peptide antigen reagent may be detected. In general, ELISA assays and immunosorbent assays, including ELISA membrane-based immunosorbent assays, as well as variations of these assays, are known and practiced by those having skill in the art.

Solid or solid phase substrates, or carriers, that can be effectively used in such assays are well known to those of skill in the art and include, for example, 96-well (or greater) microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in U.S. Patent Application Publication No. US 2010/0093557 A1. Methods for the automation of immunoassays are well known in the art and include, for example, those described in U.S. Pat. Nos. 5,885,530, 4,981,785, 6,159,750 and 5,358,691, which are incorporated by reference herein in their entirety.

In an embodiment, a multiplex assay, such as a multiplex ELISA, can be used to detect simultaneously different specific antibodies in a test sample. In embodiments, such methods employ an array, wherein multiple binding agents (for example capture antibodies) specific for multiple antibodies are immobilized on a substrate, such as a membrane, with each capture agent being positioned at a specific, pre-determined, location on the substrate. Methods for performing assays employing such arrays include those described, for example, in U.S. Patent Application Publication Nos. US 2010/0093557A1 and US 2010/0190656A1, the disclosures of which are specifically incorporated by reference herein. If flow cytometry, chemiluminescence, or electron-chemiluminescence technology is employed, multiplex arrays can be used in several different formats. Illustratively, flow cytometric multiplex arrays, also known as bead-based multiplex arrays, include the Cytometric Bead Array (CBA) system from BD Biosciences (Bedford, MA) and multi-analyte profiling (xMAP®) technology from Luminex Corp. (Austin, TX), both of which employ bead sets which are distinguishable by flow cytometry.

In another embodiment, a multiplex ELISA from Quansys Biosciences (Logan, UT) involves coating multiple specific capture reagents at multiple spots (one reagent at one spot) in the same well on a 96-well microtiter plate. Chemiluminescence technology is then used to detect multiple antibodies that bind at the corresponding spots on the plate.

In certain embodiments, a patient can be diagnosed for a fungal-related disease by adding a biological sample (e.g., blood or serum) from the patient to the kit, or components thereof, and detecting the relevant sample antibodies that specifically bind to the recombinant kex peptide antigen reagents from Histoplasma capsulatum, Coccidioides immitis, Mucor circinelloides, Rhizopus oryzae (also called Rhizopus arrhizus), Rhizopus delemar, or Candida auris fungal pathogens. By way of example, the method involves (i) collecting a blood or serum sample from the subject; (ii) adding the subject's sample to the components in the kit, e.g., a holding tube or a substrate; and (iii) detecting the fungal kex peptide antigens to which the sample antibodies bind. In this method, the subject's sample, e.g., blood or serum, is brought into contact with the kex peptide antigen reagent(s). If specific anti-kex peptide antigen antibody(ies) are present in the sample, such antibodies will specifically bind to the kex peptide antigen reagents, or a subset thereof. In other kit and diagnostic embodiments, blood is not collected from the patient (i.e., it is already collected), and is assayed for the presence of antibodies against the non-naturally occurring, recombinant fungal kex peptide antigen. In some embodiments, antibodies generated against the kex peptide antigens cross-react with one, one or more, two or more, three or more, four or more, or five or more of the kex peptide antigens of Histoplasma capsulatum, Coccidioides immitis, Mucor circinelloides, Rhizopus oryzae (also called Rhizopus arrhizus), Rhizopus delemar, or Candida auris fungal organisms, e.g., the kex peptides of SEQ ID NOs: 1-6, using the kit. In other embodiments, the sample may comprise a tissue sample or a clinical sample, which can be processed, e.g., homogenized and/or suspended in medium or buffer, prior to assay. In embodiments, any antibody(ies) found to be present in a test sample from a subject may be isolated, or isolated and purified, and further characterized.

The kit can also comprise a washing solution or instructions for making a washing solution, in which the combination of the capture reagents and the washing solution allows for capture of anti-kex peptide antigen antibodies on the solid support for subsequent detection by, e.g., secondary antibodies, labeled kex peptide reagent, or mass spectrometry. In a further embodiment, a kit can comprise instructions for suitable operational parameters in the form of a label or separate insert (package insert). For example, the instructions may inform a consumer or user about how to collect the sample, how to wash the anti-kex peptide antibody and kex peptide antigen reagent complex after binding has occurred, how to interpret the results, etc. In yet another embodiment, the kit can comprise one or more containers with appropriate positive and negative controls or control samples, to be used as standard(s) for detection, calibration, or normalization.

In another aspect, kits are provided for the treatment, protection against, or prevention of disease, pathology, and/or the symptoms thereof, caused by or associated with infection by one or more of Histoplasma capsulatum, Coccidioides immitis, Mucor circinelloides, Rhizopus oryzae (also called Rhizopus arrhizus), Rhizopus delemar, or Candida auris fungal pathogens. In some embodiments, the kit includes an effective amount of a therapeutic or prophylactic antiserum, which contains anti-kex peptide antigen antibodies or antigen binding fragments thereof that bind to/react with the non-naturally occurring, recombinant kex peptide antigen of one or more of Histoplasma capsulatum, Coccidioides immitis, Mucor circinelloides, Rhizopus oryzae (also called Rhizopus arrhizus), Rhizopus delemar, or Candida auris. In some embodiments, the cross-reactive antibodies are in unit dosage form. In an embodiment, the antiserum is an isolated antiserum. In other embodiments, the kit includes a therapeutic or prophylactic pharmaceutical composition containing an effective amount of an anti-fungal immunoprotective agent, such as a recombinant, immunogenic kex peptide antigen of Histoplasma capsulatum, Coccidioides immitis, Mucor circinelloides, Rhizopus oryzae (also called Rhizopus arrhizus), Rhizopus delemar, or Candida auris, or an antiserum containing antibodies generated against such recombinant, immunogenic kex peptide antigens, in unit dosage form. In some embodiments, the kit comprises a device (e.g., an injectable device for subcutaneous (SC) administration, a wearable injector device for SC administration, a syringe and needle, and the like, a nebulizer, or metered-dose inhaler) for dispersal of the pharmaceutical composition or a sterile container which contains the pharmaceutical composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired, a pharmaceutical composition is provided together with instructions for administering the pharmaceutical composition, e.g., containing isolated antiserum, to a subject having, or at risk of contracting or developing, a fungal infection, particularly infection and disease and the symptoms thereof caused by one or more of the Histoplasma capsulatum, Coccidioides immitis, Mucor circinelloides, Rhizopus oryzae (also called Rhizopus arrhizus), Rhizopus delemar, or Candida auris fungal organisms. The instructions will generally include information about the use of the composition for the treatment, protection against, or prevention of disease, pathology, and/or symptoms thereof associated with or caused by infection by one or more of the Histoplasma capsulatum, Coccidioides immitis, Mucor circinelloides, Rhizopus oryzae (also called Rhizopus arrhizus), Rhizopus delemar, or Candida auris fungal organisms. In other embodiments, the instructions include at least one of the following: description of the therapeutic/prophylactic agent; dosage schedule and administration for treatment, protection against, or prevention of a disease, pathology, and/or the symptoms thereof caused by one or more of Histoplasma capsulatum, Coccidioides immitis, Mucor circinelloides, Rhizopus oryzae (also called Rhizopus arrhizus), Rhizopus delemar, or Candida auris fungal organisms; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the presently described aspects and embodiment of the disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides such as those described herein, and, as such, may be considered in making and practicing the aspects and embodiments described herein. Particularly useful techniques for particular embodiments will be discussed in the sections that follow.

### EXAMPLES

The following examples are put forth to provide those of ordinary skill in the art with a complete disclosure and description of how to make and use the assay, screening, and therapeutic methods as described herein, and are not intended to limit the scope of the described aspects and embodiments.

### Example 1: Identification of Pan-Fungal Consensus Sequences of Kex1

To identify pan-fungal peptide 2 (SEQ ID NO: 7), multisequence alignments of KEX1 peptide sequences from Pneumocystis (Accession No. EU918304.1) (isolated from macaque) Aspergillus fumigatus kexin (Accession no. XM746441), Candida albicans kexin (Accession no. AF022372), and Cryptococcus neoformans kexin (Accession no. XP572303.1) were performed using Clustal Omega (http://www.ebi.ac.uk/Tools/msa/clustalo/) to analyze sequence identity and similarity. The 90-mer pan-fungal peptide 2, a pan-fungal consensus KEX1 peptide as set forth in SEQ ID NO: 7, resulted from the analysis. A DNA sequence encoding the Pan-fungal 2 consensus kex1 peptide was cloned in an E. coli expression vector and the recombinant protein was produced, isolated and purified.

### Example 2: Generation of Recombinant Kexin Proteins or Kex Peptides

Amino acid sequences of the kex peptides of the various fungal organisms described herein, as well as pan-fungal kex peptide 2, were back-translated (www.ebi.ac.uk/Tools/st/emboss_backtranseq/) with an Escherichia coli K-12 codon bias and inserted into the expression vector pET-28b(+) using NcoI and BamHI restriction sites (GenScript). Pneumocystis kex1 was cloned using the Genbank: EU918304.1 sequence. Each insert contained an additional GC 5′ to the kex sequences followed by CG to keep the kex sequence inserts in frame. Plasmids were transformed into Escherichia coli BL21 (DE3) cells and plated on LB agar supplemented with 40 μg/mL kanamycin to select for transformed clones. All recombinant kexin proteins were expressed and purified as described in Example 3 below.

### Example 3: Procedures for the Purification of Recombinant Kexin Proteins or Kex Peptides

This example describes a protocol for purifying recombinantly produced (pET28b vector), (Millipore-Sigma, US), kex polypeptides or peptides of various fungal origin that are histidine tagged. Elution conditions differ for Aspergillus kex and Cryptococcus kex proteins due to the insoluble nature of these recombinant proteins.

### Materials and Equipment

A. LB (Lysogeny Broth) growth medium with kanamycin (40 μg/mL), typically in a 1 L volume, pH to 7.5. 10 g NaCl, 5 g Yeast Extract, and 10 g Tryptone Peptone are admixed; the volume is brought to 1 L with distilled/deionized H₂O.
B. 1 M IPTG solution.
C. Extraction buffer: (sterile filtered), 50 mM Sodium Phosphate, 300 mM NaCl, 10 mM imidazole, 6 M Guanidine-HCl, pH 7.4.
D. Wash buffer: (sterile filtered) 50 mM Sodium Phosphate, 300 mM NaCl, 10 mM imidazole pH 7.4.
E. 1 M imidazole solution in 50 mM Sodium Phosphate, 300 mM NaCl, pH 7.4) and 0.2 μm sterile filter (sterile filtered).
F. HisPur™ Cobalt resin (ThermoFisher #89966)
G. Disposable 5 mL polypropylene column (Thermo P #29922).
H. His-tag protease inhibitor cocktail (PIC) (Sigma P #8849).
I. Bradford Dye (Bio Rad P #500-0006).
J. Bovine Serum Albumin Standard (ThermoFisher #23209).
K. Coomassie Blue stain containing 0.2% Coomassie Blue, 7.5% acetic acid and 50% ethanol.
L. Coomasie Blue destain containing 50% methanol, 10% acetic acid and 40% dH₂O.
M. Acrylamide Bis 30% (Sigma P #1001356385)
N. N,N,N′,N′-Tetramethylethylenediamine (TEMED) (Sigma P #1001434505).
O. Sodium dodecyl sulfate (SDS) (10% stock solution).
P. Ammonium persulfate (APS) (10% stock solution).
Q. 1.5 Tris buffer pH 8.8 (187 g Tris Base into 1 L dH₂O, bring pH to 8.8).
R. 0.5 Tris buffer pH 6.5 (60.5 g Tris Base into 1 L dH₂O, bring pH to 6.5).
S. Spectra™ Multicolor Ladder-Broad range stained (Thermo P #22634).
T. SDS-PAGE sample buffer (4×).
U. Hoefer gel casting system (model SE250).
   The procedure used is as follows:
A. Culture and induce protein expression in E. coli:
   a. Streak kexin construct on pET28b(+) onto LB KAN agar plate and incubate at 37° C. overnight (O/N) or at RT on the bench top until adequate bacterial growth/colonization is obtained. (Plates can be stored at 4° C. for ~1 month)
   b. Inoculate a single colony into 10 mL liquid LB KAN 40 (10 μL of 40 mg/mL KAN per 10 mL LB), (allowing ~1:5 liquid to air ratio), and grow at 37° C. overnight with shaking.
   c. Following overnight incubation, dilute culture 1:50 into liquid LB KAN 40 (4 mL of overnight culture into 196 mL of fresh medium) and leave at 37° C. on shaker.
   d. Grow cultures to an OD₆₀₀ 0.5 and then add 1 mM IPTG to induce expression; leave at 37° C. on shaker for 4-5 hours.
   e. Split total volume of culture among five 50 mL Oakridge tubes (~40 mL culture per tube). For scaling up: can use 250 mL Oakridge tubes for larger volumes.
   f. Harvest cells by centrifugation at 6,000×g and 4° C. for 25 minutes (Can use SS-34 or SLA-1500 rotor).
   g. Pour off supernatant and freeze cell pellets at −80° C. until time of use. Do not store E. coli pellets for longer than two weeks prior to protein extraction.
B. Protein purification using Talon metal affinity resin:
   a. Thaw pellet on ice and re-suspend cell pellet in 10 mL extraction buffer+200 μL PIC.
   b. Incubate at 4° C. for 2 hours minutes on nutator.
   c. Centrifuge suspension at 10,000×g and 4° C. for 20 minutes (use SS-34 rotor).
   d. Collect supernatant and keep on ice until Talon resin is prepared.
   e. Prepare polypropylene elution column by suspending column in the upright position; adding a few drops of wash buffer to a porous disc, then using reverse end of a Pasteur pipette to depress disc evenly to the bottom of the column.
   f. Prepare Talon resin: Resuspend Talon resin by gently shaking and add 3.5 mL of resin to a 15-mL conical tube and spin for 5 minutes at 500×g. Carefully remove ethanol layer without disturbing resin. Add 10 mL of deionized water to wash resin and spin again for 5 minutes at 500×g. Remove supernatant carefully and discard. Equilibrate resin in 10 mL of extraction buffer and spin for 5 minutes at 500×g. Remove supernatant carefully and discard.
   g. Batch bind clarified lysate and equilibrated resin by mixing together and nutating for 1 hour at 4° C.
   h. Add lysate and resin suspension to the prepared polypropylene elution column. Discard flow through.
   i. Wash resin with 15 column volumes of extraction buffer followed by 15 column volumes of wash buffer.
   j. Elution of Pneumocystis kex1, Candida kex, and Pan-fungal peptide 2.
   Elute in 1.5 mL fractions with increasing imidazole concentration in wash buffer and collect elution fractions.
      Elution gradients
      a. Add 1.5 mL of 75 mM imidazole in wash buffer and collect fraction.
      b. Add 1.5 mL of 100 mM imidazole in wash buffer and collect fraction.
      c. Add 1.5 mL of 125 mM imidazole in wash buffer and collect fraction.
      d. Add 1.5 mL of 150 mM imidazole in wash buffer and collect fraction.
      e. Add 1.5 mL (×2) of 175 mM imidazole in wash buffer and collect fraction.
      f. Add 1.5 mL (×3) of 200 mM imidazole in wash buffer and collect fraction.
      j. Elution of Pneumocystis kex1, Pan-fungal peptide 2.
   k. Elution of Aspergillus kex and Crytococcus kex (Insoluble proteins)
      a. Resuspend resin in 1.5 mL of 1% SDS and transfer into two 1.5 mL tubes.
      b. Boil suspension in a heating block 95° C.
      c. Centrifuge suspension for 5 minutes at 500×g.
      d. Collect supernatant fraction containing denatured protein.
      e. Repeat steps a-d three times to recover additional protein.
   l. Add 20 μL PIC to each fraction of interest and store at 4° C. (Imidazole solutions should be stored on ice prior to use.)
C. Quantification of protein in elution fractions (Bradford Assay-low concentration standard curve):
   a. Remove BSA-100 μg/mL from freezer (4° C.) and thaw on ice.
   b. Set up cuvettes for standard curve and add the specified amounts of both the thawed BSA/dH₂O from the below table (Table 1).

TABLE 1

| Final Conc. (μg/mL) | dH₂O (μL) | BSA-100 μg/mL (μL) |
|---|---|---|
| 0 | 200 | 0 |
| 0.25 | 197.5 | 2.5 |
| 0.5 | 195 | 5 |
| 1 | 190 | 10 |
| 2 | 180 | 20 |
| 4 | 160 | 40 |
| 8 | 120 | 80 |

c. Add an additional cuvette for each fraction and dilute samples 1:50 (20 μL sample+180 μL dH₂O).
d. Prepare Bradford dye 1:4 in dH₂O (10 mL dye+30 mL dH₂O) and add 800 μL to each cuvette (final volume 1 mL).
e. Mix cuvettes individually via inversion and incubate at RT for 15 minutes.
f. After incubation, add 200 μL of the 0 μg/mL BSA standard in replicate to wells A1 and A2 of 96 well flat bottom plate followed by the addition 0.25 μg/mL BSA standard to B1 and B2. Continue to add the BSA standard in increasing concentration to the plate in the same order.
g. Once the entirety of the BSA standard is added to the plate, load samples in replicate into the wells immediately below until no rows remain and then proceed to the top row of the next two columns.
h. After all samples are loaded onto the plate read at 595 nm-“Low-conc. Std. Curve.”
i. Record the linear regression (R2) and BSA standard curve values (Data obtained from assays with R2<0.95 should not be used). Raw values for samples represent a 1:50 dilution and should therefore be multiplied by 50 in order to convert back into μg/mL. Once the concentrations of protein have been determined, fractions intended for plate coating (e.g., ELISA/ELISPOT), injection, etc. must be electrophoresced on a 15% 2 mm SDS-PAGE gel to evaluate purity.
D. Identification of protein via SDS-PAGE gel Coomassie Blue staining:
   a. For each gel, wash 1× glass cover plate, 1× white aluminum backing plate, 2× black plate spacers, and 1× white 10 lane stacking comb with dH₂O. Rinse with 70% ethanol solution. Confirm that all solidified gel residue from previous use is removed before casting.
   b. After all materials have air-dried, take the backing plate and lay it flat on the bench top and place a spacer on each side of the plate before sandwiching with the clear glass cover plate. Confirm that the notches of the spacer are properly aligned to the edges of both plates.
   c. Loosen all screws on the casting block and slide the sandwiched plates with spacers into the caster. Confirm that all plates and spacer are even and aligned. Leave ~3 mm of the sandwiched plates protruding from the bottom of the casting block before carefully tightening the screws so as not to crack the plates.
   d. Place the casting block into the holder and set the black plastic plugs into the holder. Turn plugs to depress casting block into the black rubber mat of the holder. Confirm that the bottom of the plates is well sealed by the rubber of the holder in order to avoid leaks.
   e. Prepare separating/running gel according to the recipe below for a 15%—SDS PAGE gel and add solution to the cavity between aluminum backing plate and glass cover plate. Allow ~1.5-2 cm of space at the top of the sandwiched plates for stacking gel. Add ~1 mL of dH₂O to casting block. The gel will begin to polymerize once the APS/TEMED are added to the solution.

| Separating Gel | 15% | |
|---|---|---|
| Acrylamide Bis 30% | 5 | mL |
| Water (dH₂O) | 2.34 | mL |
| 1.5 Tris Buffer pH 8.8 | 2.5 | mL |
| SDS (10%) | 100 | μL |
| APS (10%) | 50 | μL |
| TEMED | 10 | μL |

f. Once the gel has hardened (approximately 35 minutes), remove the layer of water and prepare the stacking gel solution from the recipe below. Add solution quickly.

| Stacking Gel | 15% | |
|---|---|---|
| Acrylamide Bis 30% | 700 | μL |
| Water (dH₂O) | 3.2 | mL |
| 0.5 Tris Buffer pH 6.5 | 1250 | μL |
| SDS (10%) | 50 | μL |
| APS (10%) | 60 | μL |
| TEMED | 20 | μL |

g. Immediately place the white 10 lane stacking comb into the stacking gel and allow to fully polymerize (about 10 minutes).
h. Prepare samples to electrophoresce on gel: (5 μg protein per well)
   i. If the fraction concentration is <165 μg/mL, use 22.5 μL of sample+7.5 μL 4× Sample Buffer;
   ii. If the fraction concentration is >165 μg/mL=>sample vol.=5 μg/(conc. (μg/mL)/1000) & 4× Sample Buffer vol.=(⅓)*sample vol.
i. Heat-inactivate all samples for 10 minutes at 56° C. in the water bath.
j. Remove 15% SDS-PAGE gel from casting block and attach to the running apparatus with 2× red clips. Fill the cavity of the running apparatus and the bottom tray with 1× SDS-PAGE running buffer. The stacking comb can now be removed. Add 10 μL of the Broad Range stained (P #26634) SPECTRA™ Multicolor Ladder to first well of the gel followed by 30 μL of the prepared samples to the subsequent wells.
k. Once all samples are loaded, attach the electrodes to their appropriate terminals and turn on the power supply (red to red, black to black). Allow the gel to run at ~80-120 volts for 1.5-2.5 hours until the dye band runs of the bottom of the gel. At that point turn off the machine and disconnect the electrodes (Note: Lower voltages and lower time intervals increase the quality of the resulting gel).
l. Drain the running buffer from the running apparatus. Remove the red clips, spacers, and gently detach the glass cover plate from the gel casting frame. Use the hard plastic straight edge of the gel scraper to cleave the stacking gel off and into the trash. Divide gel as necessary for further assays, i.e. Western Blot, etc. (it is not necessary to notch a corner of the gel to establish orientation because of the stained ladder used).
m. For the separating gel that will be stained, wash 3× w/dH₂O for 15 minutes. Add ~25 mL of Coomassie Blue stain to the gel for 2+ hours or overnight if necessary. Destain with Coomassie Blue de-stain until optimal band color/gel transparency is obtained. Take a picture and save as JPG/TIF file.

### Example 4: Generation of Immune Sera

Immunization of Rhesus Macaque with Recombinant Pan-Fungal Peptide 2

An adult Rhesus macaque (monkey subject number 2116) was intramuscularly immunized with 100 μg of pan-fungal peptide 2. The monkey received a booster at 8 weeks post administration and was infected with SIV 8 weeks post boost. ELISA analysis of serum samples collected from monkey subject number 2116 was used to determine the reciprocal endpoint titers (RET) with pan-fungal peptide 2, Cryptococcus kex, Pneumocystis kex1, and Aspergillus kex as targets. As used herein, RET is the reciprocal of the highest analyte (e.g., antibody or antiserum) dilution that gives a reading above the cutoff value. For the RET analyses, serum obtained from the test animal is assayed to determine the presence of immunoglobulin (IgG) that is directed to, binds to, and/or has activity against the target protein(s), (kex peptide antigens/recombinant kex peptide antigens).

### Example 5 Western Blot Analysis Demonstrating Antiserum (Antibody) Cross-Reactivity

Animals (mice and non-human primates (NHPs)) were immunized with an immunogenic vaccine containing pan-fungal peptide 2 as immunogenic antigen. Western blot analysis was used to assess the cross-reactivity of vaccine-generated anti-pan-fungal peptide 2 antibodies with proteins that had been previously established as protective vaccine candidates. (Kling, H. M. et al., 2016, J. Infect. Dis., 213(10):1586-1595; Kling, H. M. et al., 2010, Infect. Immun., 78(10):4320-4330). In order to minimize any cross-reactivity attributable to the linker or histidine tags utilized in the KEX1 recombinant constructs, NHPs were vaccinated with a modified pan-fungal peptide 2 recombinant protein lacking any vector-associated amino acids and aluminum hydroxide.

The cross-reactivity of vaccine generated, anti-pan-fungal peptide 2 antibodies to other fungal kex1 proteins/peptides was evaluated. Purified, recombinant pan-fungal peptide 2 peptide, and purified, recombinant 90-mer kex1 peptides from Histoplasma capsulatum (HC.KEX1; accession number EER45430.1, residue numbers 316 to 402), Mucor circinelloides (MC.KEX1; accession nnumber EPB86942.1, residue numbers 290 to 378), Coccidioides immitis (CI.KEX1; XP_572303.1, residue numbers 319 to 405), Candida auris (CAu.KEX1; accession number NW_017263955.1, residue numbers 304 to 393), and C. neoformans (CN.KEX1) were electrophoresced on SDS-PAGE gels, transferred to a nitrocellulose membrane, blocked in 5% nonfat milk, and incubated with plasma obtained from pan-fungal peptide 2-immunized NHPs. Blots were washed, incubated with horseradish peroxidase-conjugated IgG secondary antibody, and developed according to standard protocols. Western blot intensity was quantified by ImageJ according to NIH guidelines and presented relative to the intensity of anti-modified pan-fungal peptide 2 plasma on a pan-fungal peptide 2 band (Schneider, C. A. et al., 2012, Nat. Methods, 9(7):671-675). Antisera antibody cross-reactivity as observed from the Western blot analysis is demonstrated in FIGS. 1A and 1B.

The relative intensity of cross-reactivity of the anti-pan-fungal peptide 2 antibodies assessed by western blotting and quantified by ImageJ correlates with the degree of sequence identity between the consensus pan-fungal peptide 2 amino acid sequence and the amino acid sequences of the species-specific-kex1 peptides of the other fungal organisms evaluated, e.g., SEQ ID NOs: 1-6. (P=0.0009, FIG. 1C).

### Example 6: Kex Peptide Enzyme Linked Immunosorbent Assay (ELISA)

This example describes a protocol for performing an ELISA immunoassay utilizing the pan-fungal peptide 2, or a kex peptide antigen derived from one or more of the fungal pathogens described herein, namely, Histoplasma capsulatum, Coccidioides immitis, Mucor circinelloides, Rhizopus oryzae (also called Rhizopus arrhizus), Rhizopus delemar, or Candida auris. The ELISA is conducted to detect (and quantify) the presence of anti-fungal kex peptide antigen antibodies in a sample, e.g., blood, plasma, serum, bronchoalveolar lavage, or biological fluid sample. The anti-kex peptide antigen antibodies to be detected (and quantified) can be directed against, reactive with and/or bind to pan-fungal peptide 2 or to one or more of the kex peptide antigens of one or more of the differemt fungal pathogens Histoplasma capsulatum, Coccidioides immitis, Mucor circinelloides, Rhizopus oryzae (also called Rhizopus arrhizus), Rhizopus delemar, or Candida auris.

### Materials and Equipment

A. KEX1 protein, which may be purified as described in Example 2
B. 1×PBS
C. Immulon high-binding (4HBX) Flat bottom microtiter plates (Thermo #3855)
D. Blocking buffer: 5% skim milk in 1×PBS
E. Wash buffer: 1× Phosphate-buffered Saline (PBS)+0.05% Tween-20
F. Secondary Antibody: Goat anti-human immunoglobulin-conjugated horseradish peroxidase (1:10,000 for IgG; Sigma-Aldrich).
G. Normal human plasma (Atlanta Biologicals, Inc., Lawrenceville, GA).
   Negative/normal control plasma with undetectable absorbance at OD₄₅₀ (i.e., equal to or less than dilution buffer alone) in KEX-ELISA at a dilution of 1:100 is used as negative controls.
H. Substrate: 3,3′,5,5′-Tetramethylbenzidine (TMB) peroxidase substrate (such as SureBlue TMB substrate, 1-component; KPL, Inc.)
I. Stop solution: 1M H₂SO₄
J. Adhesive sealing film for microplates (Plate sealers) (such as SealPlate non-sterile films from Excel Scientific, cat #100-SEAL-PLT)
K. 96-well plate reader (any system capable of reading OD at a wavelength of 450 nm). The procedure for performing the ELISA is as follows:
A. Coating/blocking ELISA plates with kex1 protein:
   a. Prepare mkkex protein in 1×PBS at 5 μg/mL. Add 50 μL of diluted KEX1 per well of Immulon 4HBX flat-bottom ELISA plates. Cover plates tightly with Parafilm or plate sealers and incubate overnight at 4° C.
   b. Following overnight incubation, remove buffer by flicking into sink or bucket and tap plate onto absorbant pad or paper towels to remove excess. Wash plates 2× with wash buffer (PBS 0.05% Tween-20) (approximately 200 μL wash buffer per well for each wash, flicking and tapping plate between washes).
   c. Add 100 μL of blocking buffer (5% milk/PBS) to each well and incubate for 1 hour at 37° C.
   d. Empty plates, wash 2× with wash buffer. The plates can be sealed and frozen at −20° C. at this step, until ready for use.
B. Handling of plasma or other infectious fluids (e.g., bronchoalveolar lavage (BAL) fluid supernatant, etc.)—First-time use.
   a. Remove plasma aliquot from −80° C. freezer.
   b. Option 1: Heat-inactivate entire aliquot at 56° C. for 30 minutes.
      Option 2: If heat inactivation of the plasma sample would be detrimental to other potential uses, thaw sample at 4° C. or on ice. Remove an aliquot (~100 μL), transfer to a new tube, and heat inactivate (30 minutes, 56° C.). Return the remaining sample to the −80° C. freezer, noting that it has been thawed 1×.
   c. Centrifuge sample at >10,000×g for 1-2 minutes to pellet aggregates prior to use.
   d. To prevent contamination in storage, add ~0.01 to 0.02% NaN3. Store sample aliquot for up to 6 months at 4° C. For subsequent assays, no further heat inactivation is needed; however, the sample should be centrifuged briefly prior to each use.
C. ELISA for endpoint titer determination (plasma):
   a. Dilute plasma 1:100 in blocking buffer. Add 50 μL of diluted plasma and make serial 2× (or 4×, if needed) dilutions directly in the plate (final volume in each well should be 50 μL) for generation of endpoint titers. Perform assay in duplicate; set up enough plates for all isotypes of interest, e.g., if there are 10 samples and endpoint titers are to be generated for both IgG and IgM-KEX1 antibodies, this would require setting up 4 plates (duplicate plates for both IgG and IgM). Include a negative/normal control on each plate. Cover plates with plate sealers and incubate overnight at 4° C.
   b. Empty plate (flicking and tapping), wash 4× w/wash buffer.
   c. Add 50 μL of secondary antibody (diluted in block) to each well (see appropriate dilutions under Materials and Equipment above). Incubate 1 hour at 37° C.
   d. Empty the plate and wash 6× with wash buffer.
   e. Add 100 μL of TMB to each well, protect from light and incubate for 30 minutes at 37° C.
   f. Add 25-50 μL of stop solution (1M H₂SO₄) to each well.
   g. Read OD of plates (on any standard plate reader) at 450 nm within 20 minutes of adding stop solution.

The majority of healthy adults (both humans and non-human primates) have circulating antibodies to Pneumocystis; therefore, when selecting a control sample to be used for calculating endpoint titers, plasma samples must be screened from healthy donors to determine and obtain an appropriate control. In plasma from an appropriate normal/negative control, the KEX1 OD₄₅₀ at a 1:100 dilution should be not more than 0.1; however, the lower the OD of the normal/negative control plasma, the better the control is. To control for plate-to-plate variability, the same normal/negative control should be used on all plates following the selection of an appropriate normal/negative control.
D. ELISA for endpoint titer determination (BAL Supernatant).
   a. Dilute BAL supernatant 1:100 in normal saline.
      i. Determine the urea concentration of the BAL supernatant and corresponding plasma sample using QuantiChrom Urea assay (BioAssay Systems Cat #DIUR-500).
         1. Follow instructions on kit insert, diluting plasma 1:10 in distilled water and using BAL supernatant without dilution.
         2. Plate plasma samples in the wells of a 96-well plate adding 5 μL of standard (1:10 dilution), blank (distilled water) and sample (1:10 dilution) in duplicates.
         3. Plate BAL supernatant in the wells of a 96-well plate adding 50 μL of standard (diluted to 5 mg/dL), blank (distilled water) and sample (undiluted).
         4. Add 200 μL working reagent (included in kit) and tap lightly to mix.
         5. Incubate plasma plate for 20 minutes at room temperature (RT) and read at OD₅₂₀ on a spectrophotometer.
         6. Incubate BAL supernatant plate for 50 minutes at RT and read at OD₄₃₀.
         7. Calculate urea concentrations ([urea]) for plasma and BAL supernatant as follows: [urea]=(ODsample−ODblank)/(ODstandard−ODblank)*[standard]. The concentration of standard for plasma will be 50 mg/dL and will be 5 mg/dL for the BAL supernatant.
         8. Calculate 1:100 dilution of BAL supernatant as follows:
         a. Find the 1:100 dilution factor of bal to plasma
         i. Dilution factor=100/(plasma [urea]/bal [urea])
         b. Calculate volumes for dilution for 500 μl total sample
         i. Volume of sample=500 μL/dilution factor
         ii. Volume of saline=500 μL-volume of sample
         9. Add the volume of sample and volume of saline to make 1:100 diluted BAL supernatant sample.
   b. Add 50 μL of diluted BAL supernatant and make serial 2× (or 4×, if needed) dilutions directly in the plate with normal saline (final volume in each well should be 50 μL) for generation of endpoint titers. Perform assay in duplicate; set up enough plates for all isotypes of interest, e.g., if there are 10 samples and endpoint titers are to be generated for both IgG and IgM-KEX1 antibodies, this would require setting up 4 plates (duplicate plates for both IgG and IgM). Include a negative/normal control in each plate, as described above. Cover plates with plate sealers and incubate overnight at 4° C.
   c. Empty plate (flicking and tapping), wash 4× w/wash buffer.
   d. Add 50 μL of secondary antibody (diluted in block, see appropriate dilutions under Materials and Equipment) to each well. Incubate for 1 hour at 37° C.
   e. Empty the plate and wash 6× with wash buffer.
   f. Add 100 μL of TMB to each well, protect from light and incubate for 30 minutes at 37° C.
   g. Add 25-50 μL of stop solution (1M H₂SO₄) to each well.
   h. Read OD of plates (on any standard plate reader) at 450 nm within 20 minutes of adding stop solution.
E. Determining Endpoint Titers.
   a. Plot OD readings from each sample (at all dilutions) in Excel, or similar program, as a line graph. For the normal/negative control sample, add 0.025 to each value prior to plotting as described below.
   b. The endpoint titer is defined by the dilution at which the test sample gives the same OD reading as that of the negative control (i.e., where the lines meet). Generally, the reciprocal endpoint titer is reported; thus, if the dilution is 1:1600, the endpoint titer is reported as 1600.
   c. Calculate endpoint titers from each of the duplicate plates, to confirm that the results are consistent between plates. Acceptable error is within one dilution. If reciprocal endpoint titers (RET) from duplicate plates fall within one dilution, average the titers (e.g., when doubling-dilutions are made, and a sample from plate 1 has a RET of 1600 and the RET from plate 2 is 3200, then the average titer is 2400). If endpoint titers on duplicate plates do not fall within one dilution of each other, repeat the ELISA on one additional plate and average the 2 values which are closest.

### Example 7: Pan-Fungal Vaccination

An adult Rhesus macaque was intramuscularly immunized with 100 μg of pan-fungal 2 peptide and aluminum hydroxide (Imject Alum, ThermoScientific) mixed in a 1:1 ratio and then boosted 8 weeks later with 50 μg of the pan-fungal 2 peptide and aluminum hydroxide. At eight weeks post-boost, the monkey was infected with simian immunodeficiency virus (SIV) and challenged with Pneumocystis. Seven additional Rhesus macaques were vaccinated with a sham vaccine, which consisted of the aluminum hydroxide but did not contain any kex peptide. The percentage of monkeys diagnosed with Pneumocystis pneumonia increased steadily from week 10 to week 40 for those monkeys that received a sham vaccination. The monkey vaccinated with pan-fungal 2 peptide did not develop pneumonia.

Similarly, adult Rhesus macaque monkeys are intramuscularly immunized with 100 μg of a recombinant immunogenic kex peptide antigen of Histoplasma capsulatum, Coccidioides immitis, Mucor circinelloides, Rhizopus oryzae (also called Rhizopus arrhizus), Rhizopus delemar, or Candida auris and aluminum hydroxide (Imject Alum, ThermoScientific) mixed in a 1:1 ratio and then boosted 8 weeks later with 50 μg of the respective kex peptide antigen of Histoplasma capsulatum, Coccidioides immitis, Mucor circinelloides, Rhizopus oryzae (also called Rhizopus arrhizus), Rhizopus delemar, or Candida auris and aluminum hydroxide. At eight weeks post-boost, the monkeys are infected with simian immunodeficiency virus (SIV) and challenged with the same fungal pathogen from which the immunizing recombiant kex peptide antigen was dervied, or with a different fungal pathogen. Seven additional Rhesus macaques are vaccinated with a sham vaccine, which consists of aluminum hydroxide adjuvant but does not contain any kex peptide antigen. The percentage of monkeys diagnosed with disease or disease symptoms, (e.g., lung disease, asthma, or pneumonia) is expected to increase steadily from week 10 to week 40 for those monkeys that receive a sham vaccination.

### Statistical Analysis

All statistical analyses were performed using GraphPad Prism (GraphPad Software, La Jolla, CA). Fisher's exact test used to determine the association between pan-fungal peptide 2 immunization (vaccination) and the incidence of PCP in the vaccinated group compared with the sham-immunized group. Differences in PCP diagnosis between pan-fungal peptide 2immunized and sham-immunized cohorts were compared using a Kaplan-Meier test. The relative risk of PCP was calculated using Koopman asymptotic score. Chronic Pc colonization was evaluated by Mantel-Cox test.

### Example 8: Antibodies Generated Against the Candida auris Fungal Pathogen and Uses Thereof

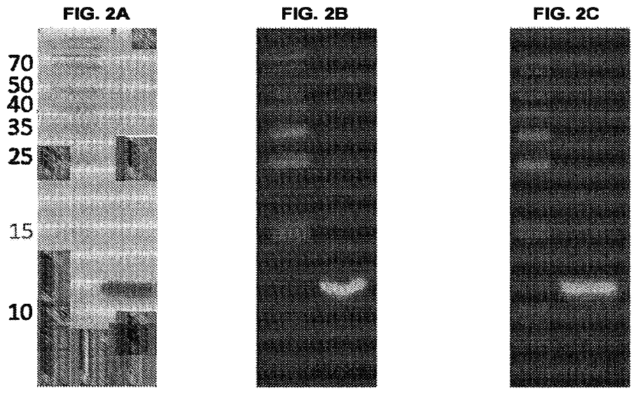

Experiments were performed to investigate the immunogenicity of the recombinant kex peptide of Candida auris (C. auris) fungal pathogen, i.e., the peptide of SEQ ID NO: 6 (called “CAu.KEX1” herein). The recombinant kex peptide was used as an immungen to immunize (e.g., administer to) both mice and non-human primates (NHPs) and to generate antiserum containing antibodies in the immunized animals. The antibodies in the sera were assessed for specific reactivity with and binding to the recombinant CAu.KEX1 peptide used as immunogen. FIGS. 2A-2C show the gel-electrophoresced recombinant kex peptide of C. auris (FIG. 2A), as well as the immunogenicity of the recombinant Cau.KEX1 peptide and the binding of antibodies in antiserum from CAu.KEX1-immunized animals to the CAu.KEX1 peptide as analyzed via western blot analysis (FIGS. 2B and 2C). The results show that the recombiannt CAu.KEX1 peptide was immunogenic and induced robust antibody production in immunized animals. In the western blot analysis, the purified protein on the blot was probed with sera from CAu.KEX1-immunized (vaccinated) mice (FIG. 2B) and from an immunized (vaccinated) NHP (FIG. 2C). The sera were diluted 1:10,000; goat anti-mouse and anti-monkey secondary antibodies were used, respectively, at a dilution of 1:10,000. FIG. 2A shows the purified CAu.KEX1 peptide electrophoresced on an SDS-PAGE gel and then stained with Coomassie Blue relative to molecular weight size markers.

### Methods

### Expression and Purification of CAu.KEX1 Peptide

CAu.KEX1 (accession number NW_017263955.1, residue number 304 to 393) cloned in pET28b+ (Novagen) in E. coli BL21 (DE3) was streaked onto LB agar plate supplemented with 40 μg/ml kanamycin and incubated at 37° C., overnight. A single colony was inoculated into 10 mL liquid LB supplemented with 40 μg/ml Kanamycin and placed on a shaker overnight at 37° C. The culture was diluted 1:50 into liquid LB supplemented with 40 μg/ml Kanamycin and placed at 37° C. for about 3 hours. Expression was induced by adding 0.5 mM IPTG and then incubated for 4 hours at 37° C. Cells were harvested by centrifuging at 6,000×g and 4° C. for 20 minutes and were stored at −80° C. overnight. Harvested cells were resuspended in 5 ml of CelLytic B Cell Lysis Reagent (Sigma) plus 1 μl of Benzonase (Sigma) plus 50 μl protease inhibitor cocktail (PIC) (Sigma) to isolate inclusion bodies. The inclusion bodies were harvested by centrifuging at 15,000 g for 10 minutes at 4° C. The inclusion bodies are then lysed with 10 ml of 6M Urea, 50 mM Sodium Phosphate and 300 mM NaCl plus 200 μl PIC (Sigma) and placed on a rocker for 2 hours. Supernatant was collected following centrifugation at 15,000 g for 20 minutes. The recombinant protein was purified by affinity chromatography using TALON resin (Takara), as per the manufacturer's instructions. The protein was eluted in elution buffer (50 mM Sodium Phosphate, 300 mM NaCl) with increasing imidazole concentration (75 mM to 500 mM). For immunization of the Rhesus macaque non-human primate (NHP), the recombinant protein with concentration 0.21 mg/ml was used for immunizaiton/vaccination. For immunization of the mice, the protein was precipitated out of solution by adding it to acidified acetone-methanol solution at a 1:4 overnight at −20° C. The concentrated protein was pelleted by centrifuging at high speed for 15 minutes before resuspending in PBS at a final concentration of 0.98 mg/ml. An endotoxin value of 6 EU/ml was observed.

### Immunization of Animals

Five (5) CD-1 mice (Charles River laboratories) were immunized subcutaneously at the base of the tail with 50 μg of the recombinant CAu.KEX1 peptide prepared 1:1 with the water: squalene adjuvant TiterMax (Sigma-Aldrich, Inc.) according to the manufacturer's guidelines. Five CD-1 mice were also sham immunized with PBS and Titermax. Blood was collected at 28 days following immunization and was centrifuged at 10000 g for 15 minutes to separate the serum component. Serum samples were aliquoted and stored at −80° C. until analyzed.

An adult Rhesus macaque (“NHP”) was immunized intramuscularly with 80 μg of recombinant CAu.KEX1 peptide prepared 1:1 with Alhydrogel (InvivoGen) according to the manufacturer's guidelines. The animal was boosted 8 weeks later with the same immunizaiton/vaccination dose. Blood was collected 2 weeks after boosting and centrifuged at 3000 g for 5 minutes to collect the serum component. The serum samples were aliquoted and stored at −80° C. until analyzed.

### Western Blot Analysis

5 μg of purified CAu.KEX1 peptide was electrophoresced on 15% SDS-polyacrylamide gels under reducing conditions. The gel was run at 100V for about 2.5 hours (hrs). One of the gels was stained with Coomassie stain. The protein from the other gel was transferred onto a nitrocellulose membrane and blocked in 5% nonfat milk overnight. The membrane (“blot”) was then incubated for 2 hrs with sera obtained from CAu.KEX1-immunized NHP and mice diluted 1:10,000 in 5% nonfat milk. The blot was then washed 3 times for 10 minutes with 1×PBS supplemented with 0.05% Tween-20 (Fisher). The blots were then incubated for an hour with either horseradish conjugated goat anti-mouse secondary IgG (ThermoFisher) or with anti-monkey secondary IgG (ThermoFisher). The blots were washed as described above before developing with West Pico PLUS Chemiluminescent Substrate (ThermoFisher). Images were taken using the Gel Imager.

### Example 9: Anti-CAu.KEX1 Antibodies Enhance Opsonophagocytic Killing of Candida auris In Vitro

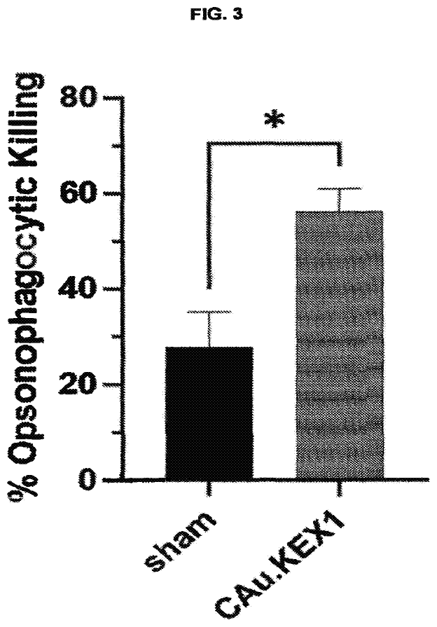

An opsonophagocytis killing (OPK) assay was performed as described by Rayens et al., 2022 (PNAS Nexus, 1(5):pgac248 (doi: 10.1093/pnasnexus/pgac248), with slight modifications. Briefly, 1×10⁵ C. auris fungal cells were pre-incubated in wells of a tissue culture plate (e.g., a microtiter plate) either with sera obtained from animals (mice) immunized with recombinant CAu.KEX1 peptide or with sera obtained from sham immunized animals (mice) for 30 minutes at 37° C. Thereafter, the fungal cells were co-cultured with LPS (1 ng/ml) activated-(24 hrs prior) murine RAW 264 macrophages to induce phagocytosis (1:1 ratio with fungal cells) in a total of 200 μl. After 2 hrs of incubation with gentle shaking, the contents of each well were quantitatively plated and were grown for 48 hrs at 37° C. on Yeast Extract-Peptone-Dextrose (YPD) agar plates. Colony forming units (CFU) were counted, and the percent killing was calculated using the following formula: 1-[CFUs from tubes with (mouse serum+fungi+macrophages/neutrophils)/average CFU in tubes with (fungi+macrophage/neutrophils)]. The results showed that antibodies in the sera (plasma) obtained from animals (mice) immunized with recombinant CAu.KEX1 peptide contained anti-CAu.KEX1 peptide antibodies that promoted opsonophagocytic killing of C. auris fungal cells compared with the sera of sham-immunized mice (FIG. 3). The serum antibodies directed against the recombinant CAu.KEX1 peptide significantly enhanced the killing of C. auris fungal cells by macrophage phagocytosis compared with serum from sham-immunized mice. (FIG. 3).

### Methods

### Opsonophagocytic Killing Assay

A volume of 95 μl of C. auris cell suspension (~2×10⁵ CFU/well) was placed the wells of a 96-well tissue culture microtiter plate. An aliquot of 10 μl of sera from CAu.KEX1-immunized mice or sham-immunized mice was added to each well, and the contents were incubated for 30 minutes at 37° C. 95 μl of a suspension of activated macrophages were then added to each well (1:1 ratio with fungal cells) in a total volume of 200 μl. After 2 hrs of incubation with gentle shaking, the contents of each well were quantitatively plated and grown for 48 hrs at 37° C. on YPD agar plates. Colony forming units (CFU) were counted, and the percent killing was calculated using the following formula: 1-[CFUs from tubes with (mouse serum+fungi+macrophages/neutrophils)/average CFU in tubes with (fungi+macrophage/neutrophils)].

### Macrophage Culture

Cells of the murine macrophage cell line, RAW 264.7 (ATCC), were cultured in complete culture medium (RPMI-1640, 10% heat-inactivated FBS, 1% penicillin, streptomycin and glutamine) (all from ThermoFisher) at 37° C. in 5% CO₂ until a sufficient number of cells was achieved. The macrophages were then activated with 1 ng/ml Lipopolysaccharide (LPS) (Sigma) for 24 hrs. The activated macrophages were harvested and adjusted to a concentration of 2×10⁶ cells/ml before use.

Preparation of Candida auris

C. auris (South Asian Clade 1, CAU-09, CDC & FDA AR Isolate Bank) fungal cells were streaked on a Yeast Extract-Peptone-Dextrose (YPD) agar plate and incubated at 30° C. for 48 hours. A single colony was inoculated into 10 ml of YPD broth and incubated overnight at 30° C. with shaking. The fungal cells were harvested by centrifuging at 1500 rpm for 5 minutes before washing 3 times with 1×PBS at 1500 rpm for 5 minutes. The fungal cells were counted and adjusted to a concentration of 2×10⁶ CFU/ml in culture medium.

### Other Embodiments

From the foregoing description, it will be apparent that variations and modifications may be made to the aspects and embodiments described herein to adopt them to various usages and conditions. Such embodiments are also within the scope of the following claims. The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An isolated, recombinant immunogenic peptide antigen or a polynucleotide encoding the peptide antigen having at least about 95% or more amino acid sequence identity to one or more of the following, or comprising or consisting of the following:
DDGMTMEGPGTLIQRAFVNGIQQGRAGRGSIYVFAAGNGALHEDNCNFD
GYTNSIYSVTVGAVDRDDNHPYYSESCSAMLVVTYSSG as set
forth in SEQ ID NO: 1;
DNGATMDAPGLLIRRAMVHGIQQGRGGKGSIFVFAAGNGAASGDNCNFD
GYTNSIYSITVGAIDREDKHPYYSESCSAQLVVTYSSG as set
forth in SEQ ID NO: 2;
PDNGQTMEAPNGILADAFINGIKKGRGGKGSIYVFATGNGATSGDNCNF
DGYTNSIYTITVGAIDHRNEHPPYSESCSAQLVVTYSSGGG as
set forth in SEQ ID NO: 3;
PDNGENMEAPKAILTDAIANGVRNGRDGKGSIYVFATGNGATLGDNCNF
DAYTNSIYTITVGAIDHTNKHPAYSESCSAQLVVTYSSGSG as
set forth in SEQ ID NO: 4;
PDLGEVAEGPQGIILDAIRNGIENGRQGKGSIFVFASGNGGHNDDNCNF
DGYTNSIYTITVGAIDRLGNYPSYAEKCAAQFFVTYSSGSG as
set forth in SEQ ID NO: 5;
and
DNGKTVAAPDMLVRKAMIKGIQDGRQGKGAVYVFASGNGRQYDDQCNF
DGYTNSIYSITVGAIDHQNQWPFYAEACSAVMVVTYSAGSG as set
forth in SEQ ID NO: 6, or immunogenic portions or
fragments thereof.

2. An immunogenic composition comprising an effective amount of the immunogenic peptide antigen of claim 1, or a polynucleotide encoding the peptide antigen, and a pharmaceutically acceptable carrier or excipient.

3. The immunogenic composition of claim 2, wherein the composition further comprises an adjuvant.

4. The immunogenic composition of claim 3, wherein the adjuvant is alpha-galactosylceramide (αGC) or alum.

5. An immunogenic vaccine comprising the isolated, recombinant immunogenic peptide antigen of claim 1, or a polynucleotide encoding the peptide antigen.

6. A method of eliciting an immune response in a subject, the method comprising administering to the subject the isolated, recombinant immunogenic peptide antigen of claim 1, or a polynucleotide encoding the peptide antigen, or a pharmaceutically acceptable composition thereof.

7. A method of treating or protecting a subject against a disease and/or symptoms thereof, associated with or caused by infection by a fungal pathogen, the method comprising administering to the subject an effective amount of the isolated, recombinant immunogenic peptide antigen of claim 1, a polynucleotide encoding the peptide antigen, or a pharmaceutically acceptable composition thereof.

8. A method of treating or protecting a subject against disease and/or symptoms thereof associated with infection by a fungal pathogen, the method comprising administering to the subject an immunogenic peptide of SEQ ID NO: 7, a polynucleotide encoding the peptide, or a pharmaceutical composition comprising the peptide or the polynucleotide, wherein the peptide elicits in the subject an immune response comprising antibodies that cross-react with a kex peptide antigen of one or more of SEQ ID NOs: 1-6 produced by different fungal pathogens.

9. The method of claim 8, wherein the fungal pathogen is selected from one or more of Histoplasma capsulatum, Coccidioides immitis, Mucor circinelloides, Rhizopus oryzae, Rhizopus delemar, or Candida auris.

10. A method of treating or protecting a subject against disease and/or symptoms thereof associated with infection by a fungal pathogen, the method comprising administering to the subject an immunogenic peptide of any one of SEQ ID NOs: 1-6, a polynucleotide encoding the peptide, or a pharmaceutical composition comprising the peptide or the polynucleotide, wherein the immunogenic peptide elicits in the subject an immune response comprising antibodies that bind to the kex peptide antigen of one or more of SEQ ID NOs: 1-6, produced by the Histoplasma capsulatum, Coccidioides immitis, Mucor circinelloides, Rhizopus oryzae, Rhizopus delemar, or Candida auris fungal pathogens.

11. An isolated antiserum comprising an antibody that specifically binds to one or more of the immunogenic peptide antigens of claim 1, or an antigen-binding fragment thereof, in an amount effective to treat or protect a subject against disease and/or a symptom thereof associated with infection by one or more of Histoplasma capsulatum, Coccidioides immitis, Mucor circinelloides, Rhizopus oryzae, Rhizopus delemar, or Candida auris fungal pathogens.

12. An isolated antiserum comprising an antibody that specifically binds to one or more of the immunogenic peptide antigens of claim 1, or an antigen-binding fragment thereof.

13. An isolated or purified antibody or an antigen-binding fragment that specifically binds to an immunogenic peptide of claim 1 in an amount effective to treat or protect a subject against disease and/or a symptom thereof associated with fungal infection by one or more of Histoplasma capsulatum, Coccidioides immitis, Mucor circinelloides, Rhizopus oryzae, Rhizopus delemar, or Candida auris.

14. The method of claim 13, wherein the method treats or protects against one or more diseases or pathologies selected from thrush, invasive candidiasis, lung or pulmonary disease or dysfunction, pneumonia-like illness, meningitis, histoplasmosis, flu-like disease, fever, chills, headache, cough, fatigue, shortness of breath, muscle aches, rash, coddidioidomycosis or valley fever, cutaneous infection, zygomycosis, and symptoms thereof.

15. The isolated antibody of claim 13, wherein the subject is treated or protected against one or more diseases or pathologies selected from thrush, invasive candidiasis, lung or pulmonary disease or dysfunction, pneumonia-like illness, meningitis, histoplasmosis, flu-like disease, fever, chills, headache, cough, fatigue, shortness of breath, muscle aches, rash, coddidioidomycosis or valley fever, cutaneous infection, zygomycosis, and symptoms thereof.

16. A method of treating or protecting a subject against disease and/or a symptom thereof associated with fungal infection, the method comprising administering to the subject an effective amount of an antibody or an antigen-binding fragment thereof that specifically binds to one or more of SEQ ID NOs: 1-6.

17. The method of claim 16, wherein the antibody or an antigen-binding fragment thereof specifically binds an immunogenic kex peptide antigen of one or more fungal pathogens selected from Histoplasma capsulatum, Coccidioides immitis, Mucor circinelloides, Rhizopus oryzae, Rhizopus delemar, or Candida auris.

18. The method of claim 13, wherein the subject is immunocompromised or immunosuppressed.

19. A method of killing or enhancing killing of a fungal cell, the method comprising
a) for Candida auris fungal cells, contacting the Candida auris fungal cells with antibodies generated against an immunogenic recombinant peptide antigen comprising or consisting of DNGKTVAAPDMLVRKAMIKGIQDGRQGKGAVYVFASGNGRQYDDQCNFDGYTNSIYS ITVGAIDHQNQWPFYAEACSAVMVVTYSAGSG as set forth in SEQ ID NO: 6, or with an antiserum containing said antibodies, in an amount effective to kill the cells;
b) for Histoplasma capsulatum fungal cells, contacting the fungal cells with antibodies generated against an immunogenic recombinant peptide antigen comprising or consisting of DDGMTMEGPGTLIQRAFVNGIQQGRAGRGSIYVFAAGNGALHEDNCNFDGYTNSIYS VTVGAVDRDDNHPYYSESCSAMLVVTYSSG as set forth in SEQ ID NO: 1, or with an antiserum containing said antibodies, in an amount effective to kill the cells;
c) for Coccidioides immitis fungal cells, contacting the fungal cells with antibodies generated against an immunogenic recombinant peptide antigen comprising or consisting of DNGATMDAPGLLIRRAMVHGIQQGRGGKGSIFVFAAGNGAASGDNCNFDGYTNSIYS ITVGAIDREDKHPYYSESCSAQLVVTYSSG as set forth in SEQ ID NO: 2, or with an antiserum containing said antibodies, in an amount effective to kill the cells;
d) for Mucor circinelloides fungal cells, contacting the fungal cells with antibodies generated against an immunogenic recombinant peptide antigen comprising or consisting of PDNGQTMEAPNGILADAFINGIKKGRGGKGSIYVFATGNGATSGDNCNFDGYTNSIY TITVGAIDHRNEHPPYSESCSAQLVVTYSSGGG as set forth in SEQ ID NO: 3, or with an antiserum containing said antibodies, in an amount effective to kill the cells;
e) for Rhizopus oryzae fungal cells, contacting the fungal cells with antibodies generated against an immunogenic recombinant peptide antigen comprising or consisting of PDNGENMEAPKAILTDAIANGVRNGRDGKGSIYVFATGNGATLGDNCNFDAYTNSIY TITVGAIDHTNKHPAYSESCSAQLVVTYSSGSG as set forth in SEQ ID NO: 4, or with an antiserum containing said antibodies, in an amount effective to kill the cells;
f) for Rhizopus delemar fungal cells, contacting the fungal cells with antibodies generated against an immunogenic recombinant peptide antigen comprising or consisting of PDLGEVAEGPQGIILDAIRNGIENGRQGKGSIFVFASGNGGHNDDNCNFDGYTNSIY TITVGAIDRLGNYPSYAEKCAAQFFVTYSSGSG as set forth in SEQ ID NO: 5, or with an antiserum containing said antibodies, in an amount effective to kill the cells.

20. The method of claim 19, wherein the contacting is in vitro, ex vivo, or in vivo.


SEQUENCE LISTING (12 sequences)

SEQ ID NO: 1 - protein, 87 aa, unidentified
DDGMTMEGPGTLIQRAFVNGIQQGRAGRGSIYVFAAGNGALHEDNCNFDGYTNSIYSVTVGAVDRDDNHPYYSESCSAMLVVTYSSG

SEQ ID NO: 2 - protein, 87 aa, unidentified
DNGATMDAPGLLIRRAMVHGIQQGRGGKGSIFVFAAGNGAASGDNCNFDGYTNSIYSITVGAIDREDKHPYYSESCSAQLVVTYSSG

SEQ ID NO: 3 - protein, 90 aa, Mucor sp.
PDNGQTMEAPNGILADAFINGIKKGRGGKGSIYVFATGNGATSGDNCNFDGYTNSIYTITVGAIDHRNEHPPYSESCSAQLVVTYSSGGG

SEQ ID NO: 4 - protein, 90 aa, unidentified
PDNGENMEAPKAILTDAIANGVRNGRDGKGSIYVFATGNGATLGDNCNFDAYTNSIYTITVGAIDHTNKHPAYSESCSAQLVVTYSSGSG

SEQ ID NO: 5 - protein, 90 aa, unidentified
PDLGEVAEGPQGIILDAIRNGIENGRQGKGSIFVFASGNGGHNDDNCNFDGYTNSIYTITVGAIDRLGNYPSYAEKCAAQFFVTYSSGSG

SEQ ID NO: 6 - protein, 89 aa, Candida sp.
DNGKTVAAPDMLVRKAMIKGIQDGRQGKGAVYVFASGNGRQYDDQCNFDGYTNSIYSITVGAIDHQNQWPFYAEACSAVMVVTYSAGSG

SEQ ID NO: 7 - protein, 90 aa, synthetic construct
PDDGKTMEGPDILVLRAFINGVQNGRDGKGSIYVFASGNGGGFEDNCNFDGYTNSIYSITVGAIDRKGLHPSYSEACSAQLVVTYSSGSG

SEQ ID NO: 8 - protein, 90 aa, unidentified
DDDGKTVDGPSPLVLRAFINGVNNGRNGLGSIYVFASGNGGIYDDNCNFDGYANSVFTITIGGIDKHGKRFAYSEACSSQLAVTYAGGSA

SEQ ID NO: 9 - DNA, 270 bp, unidentified
  source 1..270: mol_type = unassigned DNA
gatgacgatggaaaaaccgttgatgggccttctcctcttgttcttagagcatttattaatggagtaaataatgggaggaatgggttgggttctatctatgtttttgcatcaggaaatggcggaatatacgatgacaactgtaattttgatggatatgcaaatagcgtgtttactattactattggtggtatagataaacacggaaagcgctttgcatattctgaagcgtgttcttctcagttagctgttacatatgcaggcggaagtgca

SEQ ID NO: 10 - protein, 844 aa, unidentified
MRFLGSIALVLSSISVASANVRSRSYDTHEFFALHLDDSASPSHVAQLLGARHEGQIGELANHHTFSIPRERSSDLDALLERARAARKIRRRARDDATSQEQHNDALGGILWSQKLAPKKRLVKRVPPPERLARTFATGKEDPVAAQSQKRIASTLGITDPIFNGQWHLFNTVQLGHDLNVTGVWMEGITGKGVTTAVVDDGLDMYSNDLKPNYFPEGSYDFNDHTPEPRPRLSDDKHGTRCAGEIAAARNDVCGVGVAYDSRVAGVRILSKAIDDADEATAINFAYQENDIFSCSWGPPDDGATMEGPGILIKRAFVNGVQNGRGGKGSIFVFAAGNGASFEDNCNFDGYTNSIYSITVGAIDREGNHPSYSESCSAQLVVAYSSGSGDAIHTTDVGTDKCYSFHGGTSAAGPLAAGTVALALSARPELTWRDAQYLMVETAVPIHEDDGSWQVTKAGRKFSHDWGYGKVDAYALVQKAKTWELVKPQAWFHSPWLRVQHKVPQGDQGLASSYEVTEQMMKNANIARLEHVTVTMNVNHTRRGDLSVELRSPEGIVSHLSTTRKSDNEKAGYVDWTFMTVAHWGESGVGRWTVIVKDTNVNEFTGEFIDWRLNLWGEAIDGANQKPHPFPDEHDDDHSIEDAIVATTSVETGPTKTGVPGSTDDTINRPVNAKPVETQTPSPAETTATKLAPPAETRPAATATSSPTPPAASDSFLPSFMPTFGASKRTQIWIYAAIGSIIVFCIGLGIYFQVQRRKRILNNPRDDYDFEMIEDENALHGGNGRSGRTQRRGGELYNAFAGESDEEEPLFSDEDDEPYRDRAPSEDRLRDTSSDDRSLRHGDH

SEQ ID NO: 11 - protein, 924 aa, Candida sp.
MLPIKLLIFILGYLLSPTLQQYQQIPPRDYENKNYFLVELNTTNSQKPLIDFISHYRGHYNFEHQLSSLDNHYVFSIDKSHPHNSFLGNHNSNEYNLMKRQLGHEQDYDELISHVESIHLLPMKKLSKRIPVPIEMEDVVFDNRDDTGSDNHEATDEAHQKLIEIAKKLDIHDPEFTTQWHLINLKYPGHDVNVTGLWLEDILGQGIVTALVDDGVDAESDDIKQNFNSEGSWDFNNKGKSPLPRLFDDYHGTRCAGEIAAVKNDVCGIGVAWKSQVSGIRILSGPITSSDEAEAMVYGLDTNDIYSCSWGPTDNGKVLSEPDVIVKKAMIKGIQEGRDKKGAIYVFASGNGGRFGDSCNFDGYTNSIYSITVGAIDYKGLHPQYSEACSAVMVVTYSSGSGEHIHTTDIKKKCSATHGGTSAAAPLASGIYSLILSANPNLTWRDVQYISVLSATPINEEDGNYQTTALNRKYSHKYGYGKTDAYKMVHFAKTWVNVKPQAWYYSDIIEVNQTITTTPEQKAPSKRDSPQKIIHSSVNVSEKDLKIMNVERVEHITVKVNIDSTYRGRVGMRIISPTGVISDLATFRVNDASTRGFQNWTFMSVAHWGETGIGEWKVEVFVDDSKGDQVEINFKDWQFRIFGESIDGDKAEVYDITKDYAAIRRELLEKEKQNSKSTTTTSSTTTATTTSGGEGDQKTTTSAENKESTTKVDNSASITTSQTASLTSSNEQHQPTESNSDSDSDTDDENKQEGEEDNDNDNDNGNKKANSDNTGFYLMSIAVVGFIAVLLVMKFHKTPGSGRRRRRRDGYEFDIIPGEDYSDSDDDEDDSDTRRADDDSFDLGHRNDQRVVSASQQQRQYDRQQDEARDRLFDDFNAESLPDYENDMFKIGDEEEEEEEEEEGQQSAKAPSNSEGNSGTSTKK

SEQ ID NO: 12 - protein, 917 aa, Cryptococcus neoformans
MRTLLSLWGILLALIVPPSLALQRPQPRSYDTHAYYALELDPSISPAAALQLSKSLGVELVERIGELDGHWLVRTEGWTPEHASITKRSVSHDPILKRWEALPSSLGKKSLTPLSLKQRAKRHKSYSPRSRHSRDDRTELLYAQNELHLADPMLDQQWHLINTQMKDIELNVTGLWGRGITGEGVHVVIIDDGLDVESKDLKDNFFAEGSYDFNDHTELPIPRLKDDQHGTRCAGEIAAVPNDVCGVGVAYDSKIAGVRILSAPISDADEAAALNYAYQLNDIYSCSWGPPDDGRSMEAPDGLILKAMVNGVQKGRDGKGSVFVFAAGNGGGSDDQCNFDGYTNSIFSVTVGAVDRKGLHPYYSEMCAAMMVVAPSSGSGDHIHTTDVGKDKCSHSHGGTSAAAPLAVGVFALALSVRPDLTWRDIQHLAVRHAVFFNPDDPAWELTAAGRHFSYKYGYGKLDAGLFVEAAEKWQLVKPQTWYDSPSVYLPTTSPADVTRRQDEAADGPTSSDEETSNPPPVVEPSGSFITEDGVISTYEVTQSMLFDANFERLEHVTVRVWIDHQRRGDVEVELTSPNGVVSVLCRQRRFDNADSGFPGWKFMSLKHWDENPVGTWTIKVKDQVNPDKTGRFVAWSLQLWGESVDPALAKLWAPAEEGQPDEEQTGSNPSTTVSQKPKPTALLPGDHGEASGEATQPGLGSATAHPQPTSTTGDAGNVAEPTGPTDADADEGFFSGISNLASSSTWLAGAGAIIILSGAAIGAFFFIRARRQKRNLFGLSNNGQGARGAYEPVDDVQMSLLERGRRKFGKSKSESQGTKDLYDAFGDGPSDEEEEDLDERTALRYHDGFLEDDEPNEVGPKTEYKDEPESEPETFKDGEETVGTKDKGKGKGPSEGESGSGSSSSWQDAADEEARV